(19) United States
(12) Patent Application Publication
(10) Pub. No.: US 2023/0023669 A1
(43) Pub. Date: Jan. 26, 2023

(54) METHODS AND ARRANGEMENTS TO DESCRIBE DEFORMITY OF A BONE

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Andrew Phillip Noblett, Bartlett, TN (US); Johnny R. Mason, Bartlett, TN (US); Paul Bell, Memphis, TN (US); Haden Janda, Germantown, TN (US); Benjamin Ollivere, Nottingham (GB)

(73) Assignees: Smith & Nephew, Inc., Memphis, TN (US); Smith & Nephew Orthopaedics AG, Zug, TN (US); Smith & Nephew Asia Pacific Pte. Limited, Singapore (SG)

(21) Appl. No.: 17/790,869

(22) PCT Filed: Jan. 8, 2021
(86) PCT No.: PCT/US2021/012631

§ 371 Date: Jul. 5, 2022

Related U.S. Application Data
(60) Provisional application No. 62/958,833, filed on Jan. 9, 2020.

(51) Int. Cl. A61B 17/56; A61B 34/10

(52) U.S. Cl. CPC A61B 17/56; A61B 34/10

(57) ABSTRACT

Logic may determine how to reduce bone segments. Logic may communicate one or more images to display with at least two bone segments. Logic may identify a first reduction point and a third point on a first bone segment and identify a second reduction point and a fourth point on the second bone segment. Logic may identify a fifth point on the first bone segment and a sixth point on the second bone segment. Logic may also divide the one or more images along a line or plane between the bone segments, bring the second reduction point and the associated image segment to the first reduction point, align the line or plane of the second bone segment with a line or plane of the first bone segment. Furthermore, logic may adjust alignment and record the movement of the image segments or compare original and final positions, to determine deformity parameters.

### CROSS-REFERENCE TO RELATED APPLICATIONS

This is a non-provisional of, and claims the benefit of the filing date of, pending U.S. provisional patent application No. 62/958,833, filed Jan. 9, 2020, entitled “Methods and Arrangements to Describe Deformity of a Bone” the entirety of which application is incorporated by reference herein.

### FIELD OF THE DISCLOSURE

The present disclosure relates generally to orthopedic devices, systems, and methods to facilitate alignment of bone segments or surgical navigation associated with a bone segments, and particularly to describe a deformity of the bone.

### BACKGROUND OF THE DISCLOSURE

Orthopedic surgeons must analyze a wide variety of deformities in which two or more bone segments are displaced or not aligned properly. Some simple deformities can be resolved acutely in clinic or in the operating room. Other ailments require careful planning and more prolonged treatment.

Orthopedic deformities are three dimensional problems and are typically described quantitatively with six deformity parameters, which can be measured with medical images and clinical evaluations. The deformity parameters are usually described as anteroposterior (AP) view translation, AP view angulation, sagittal (LAT) view translation, sagittal view angulation, axial view translation, and axial view angulation. Angulation values are assessed by measuring the angular differences between the mechanical axes of two bone segments. Translation values are assessed by measuring the distances between points on each bone segment, which would be collocated if the bone segment were properly aligned and reduced. Deformity parameters are evaluated from medical images, AP and Lateral radiographs or three-dimensional (3D) imaging modalities, and clinical evaluations.

Modern medicine includes many digital tools which can assist orthopedic surgeons in aligning bone segments. Unfortunately, current digital tools for assessing orthopedic deformities can be laborious and may require specialized knowledge in order to properly identify and place the axes and corresponding points of the deformed bone segments. The methods and arrangements disclosed herein describe a graphical method for digitally correcting bone segments that is designed to improve analysis speed and be more easily understood by those minimally skilled in orthopedic deformities.

### SUMMARY OF THE DISCLOSURE

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

The present disclosure provides methods and arrangements for determining how to reduce two bone segments. Deformity determination logic circuitry may implement functionality to determine how to reduce two bone segments by implementation of code for execution on a processing circuits, logical functions implemented in circuitry, and/or the like. The deformity determination logic circuitry may communicate an image with at least two bone segments in a first plane to display to a user such as a doctor. In many embodiments, the deformity determination logic circuitry may identify a first reduction point on a first bone segment; identify a second reduction point on the second bone segment; identify a third point on a first bone segment to create a first line connected to the first reduction point; and identify a fourth point on the second bone segment to create a second line connected to the second reduction point. Deformity determination logic circuitry may also divide the image along the second line, bringing the second reduction point and the associated image segment to the first reduction point, aligning second line and the associated image segment with first line. Furthermore, the deformity determination logic circuitry may interact with a user to obtain input such as graphical input to adjust the alignment of the bone segments. The process may be repeated with a second image of the bone segments in a second plane, ideally (but not necessarily) orthogonal to the first image, in order to obtain deformity parameters that could not be calculated from the first image. In some embodiments, when using 3D models of the patient's bone segments, the deformity logic circuitry may identify three points on a first segment followed by three corresponding points on a second bone segment to create two planes in which reduction may align in a 3D environment.

In some embodiments, deformity determination logic circuitry may record the movement of the image segments, each containing a bone segment, to backcalculate deformity parameters from the final reduced state. In other embodiments, deformity determination logic circuitry may compare the original and final locations of the image segments, each containing a bone segment, to determine deformity parameters.

Further features and advantages of at least some of the embodiments of the present invention, as well as the structure and operation of various embodiments of the present invention, are described in detail below with reference to the accompanying drawings.

### BRIEF DESCRIPTION OF THE DRAWINGS

By way of example, a specific embodiment of the disclosed device will now be described, with reference to the accompanying drawings, in which:

FIG. 1A illustrates an embodiment of is a system for treating a patient;

FIGS. 1B-F illustrate embodiments of anteroposterior (AP) view and lateral (LAT) view outline images of a tibia aligned and misaligned;

FIG. 1G illustrates a 3D image with points and planes;

FIGS. 2A-I illustrate embodiments of postoperative radiographs (such as x-ray images) of a process of determining movements of two bone segments of a misaligned tibia to align the misaligned tibia by adjustment of the radiographs;

FIG. 3 depicts a flowchart of embodiments to identify movement of bone segments to align the bone segments;

FIG. 4 depicts an embodiment of a system including a multiple-processor platform, a chipset, buses, and accessories the server computer, HCP device, and the patient device shown in FIG. 1A; and

FIGS. 5-6 depict embodiments of a storage medium and a computing platform such as the server computer, HCP device, and the patient device shown in FIG. 1A and FIG. 4.

The drawings are not necessarily to scale. The drawings are merely representations, not intended to portray specific parameters of the disclosure. The drawings are intended to depict various embodiments of the disclosure, and therefore are not be considered as limiting in scope. In the drawings, like numbering represents like elements.

### DETAILED DESCRIPTION

Embodiments comprise systems and arrangements to identify or communicate a deformity of bone segments. Many embodiments facilitate identification and communication of the deformity by facilitation of manipulation of the bone segments in one or more images such as radiographs or other 2D or 3D medical images. For example, an embodiment may comprise deformity determination logic circuitry to interact with a user such as, e.g., a doctor. In such embodiments, the doctor may graphically interact with images with at least two bone segments to determine information about the deformity of the bone segments. Graphical interaction between the doctor and the images of the bone segments advantageously utilize skills to physically align bones to create a mathematical representation of the deformity of bone segments.

In some embodiments, the deformity determination logic circuitry may reside in a remote computer accessible via a network and, in further embodiments, via an application such as a web browser. In other embodiments, the deformity determination logic circuitry may reside on a local computer directly accessible by the user. In further embodiments, the deformity determination logic circuitry may partially reside on a remote computer and partially reside on a local computer.

Some embodiments may identify a first reduction point on the first bone segment based on graphical input and a second reduction point on the second bone segment graphical based on graphical input. The first and second reduction points may identify an interconnection point between the first bone segment and the second bone segment.

In some embodiments, identification of the first reduction point and the second reduction point involves a graphical selection of the first reduction point on the first bone segment and a graphical selection of the second reduction point on the second bone segment by a user. In other embodiments, identification of the first reduction point and the second reduction point may involve selection of two points on the first bone segment, selection of two points on the second bone segment, and calculation of the first and second reduction points. In such embodiments, the first and second reduction points may comprise midpoints (or other relative points) derived from the two points selected on the first bone segment and the second bone segment. For example, the user may graphically select two interconnection points between the first bone segment and the second bone segment on the first bone segment and the deformity determination logic circuitry may calculate the first reduction point as the midpoint (or other relative point) derived from the two interconnection points identified on the first bone segment. Similarly, the user may graphically select two interconnection points between the first bone segment and the second bone segment on the second bone segment and the deformity determination logic circuitry may calculate the second reduction point as the midpoint (or other relative point) derived from the two interconnection points identified on the second bone segment.

Some of these embodiments may receive one or more additional points. For instance, if the user selects a first reduction point on the first bone segment and a second reduction point on the second bone segment, the user may also identify a third point on the first bone segment and a fourth point on the second bone segment. The third and fourth points may identify a second interconnection point and a third interconnection point between the first bone segment and the second bone segment. The one or more additional points may also define a plane on the first bone segment and a plane on the second bone segment for three-dimensional (3D) images. Note that the numeric designation of the points does not necessarily identify the order of input of the points for all embodiments. For instance, a user may input the first reduction point, then the third point, then the second reduction point, and then the fourth point. In other embodiments, the user may identify two interconnection points (e.g., first and second interconnection points) on the first bone segment and then identify two interconnection points (e.g., third and fourth interconnection points) on the second bone segment to facilitate calculation of the reduction points on each bone segment. In several embodiments, the order of entry of the points can be set by default and/or by preference of the user. In other embodiments, the order of identification (selection or calculation) of the first and second reduction points and the third and fourth points may be established and required by the deformity determination logic circuitry.

With two points on the first bone segment, the deformity determination logic circuitry may draw a first line on the image between the first reduction point and the third point. Similarly, with the two points on the second bone segment, the deformity determination logic circuitry may draw a second line on the image between the second reduction point and the fourth point. In some embodiment the two lines may represent the edges of the bone segment. In other embodiments, the deformity determination logic circuitry may draw a cut line on the image through (or between) the interconnection point(s) and the reduction point on the second bone segment.

In some embodiments, the first reduction point is defined on the first bone segment and the deformity determination logic circuitry may define a line on the first bone segment based on the first reduction point without requiring a third point. Similarly, a second reduction point is defined on the second bone segment and the deformity determination login circuitry may define a line on the second bone segment based on the second reduction point without requiring a fourth point. The first line may pass through the first reduction point and the second line may pass through the second reduction point. In further embodiments, the two lines may also be placed independently of the reduction points either via user interaction or via analysis by the deformity determination logic circuitry.

In some embodiments, the deformity determination logic circuitry may place a first reduction point at the midpoint of the two interconnection points on the first bone segment. In some embodiments, the deformity determination logic circuitry may place a second reduction point at the midpoint of the two interconnection points on the second bone segment. In some of these embodiments, the deformity determination logic circuitry may use and collocate the first reduction point and the second reduction point and, in some embodiments, also collocate the two interconnection points on the first bone segment with the two interconnection points on the second bone segment. In such embodiments, the deformity determination logic circuitry may determine the translation and angulation based on the translation of and angulation about the first and/or second reduction points to collocate the first reduction point with the second reduction point and/or collocate at least one of the two interconnection points on the first bone segment with at least one of the two interconnection points on the second bone segment.

In several embodiments, the deformity determination logic circuitry may draw a mechanical axis through the first bone segment and the first reduction point. In some of these embodiments, the deformity determination logic circuitry may draw a vertical line through the first reduction point to approximate the mechanical axis of the first bone segment. In other embodiments, the deformity determination logic circuitry may calculate or otherwise determine a second axis point on the first bone segment for the mechanical axis through input from the user and/or from markers in the image of the bone segments and draw the mechanical axis through the first reduction point and the second axis point.

Several embodiments may create a copy of the image. Some embodiments may mask the portion of the original image on the second bone segment side of a cut line and mask the portion of the copied image on the first bone segment side of the cut line. Other embodiments may mask the two images differently. Further embodiments may divide the image into at least two portions. In such embodiments, a first portion may comprise the first bone segment, or a portion thereof, and a second portion may comprise the second bone segment, or a portion thereof. Hereinafter, the image portion or copied image with the unmasked portion that includes the first bone segment may be referred to as the first bone segment for discussions about, e.g., graphical manipulations of image portion that includes the first bone segment. The same is true for the second bone segment. In other words, rather than describing the translation or angulation of the portion of the image, some discussions below may describe such actions as translations or angulations of the bone segment that is included in the portion of the image being manipulated.

Many embodiments may collocate the first and the second reduction points to connect the first bone segment and the second bone segment to present a modified image to the user to determine an AP or LAT translation and an axial translation. Some of these embodiments may also collocate one or more additional interconnection points to, in effect, rotate the second bone segment in the modified image to determine an AP or LAT angulation.

Further embodiments may rotate a line between interconnection points on the second bone segment to be collinear in the modified image with a line between interconnection points on the first bone segment. Still further embodiments record each translation and/or angulation of one or both bone segments. Other embodiments compare the positions of one or both bone segments at least once, such as after the alignment of the bone segments is approved or saved, to determine each translation and angulation of the bone segments.

In some embodiments, the collocation of the first and second reduction points (by movement of one or both the images or portions of images with the two bone segments) may provide an estimate of, e.g., an AP translation or LAT translation, depending on the view represented in the image and may also provide an estimate of an axial translation. Some embodiments perform the collocation of the first and second reduction points automatically after identification of these two reduction points by a user and/or after the user inputs an indication to save the first and second reduction points. Some embodiments perform the collocation of the first and second reduction points and additional interconnection points automatically after identification of these points by a user and/or after the user inputs an indication to save the first and second reduction points and the additional interconnection points. Some embodiments collocate the first and second reduction points and rotate lines through additional interconnection points and the corresponding reduction points to be colinear automatically. Such embodiments may perform collocation and rotation after identification of these points and/or after the user inputs an indication to save the first and second reduction points and the additional interconnection points. Other embodiments receive graphical input and perform the translation based on or responsive to the graphical input.

The anatomical directions necessary to orient the calculated translations and angulations require a coordinate system be established for each image. The coordinate system can be derived from markers within the image such radiolucent markers, user input (e.g. an origin point and an axis placed on the image), required orientation of the images (e.g. medial is oriented to the right and proximal is oriented at the top of the screen), hardware orientation restrictions, or a combination.

In some embodiments, the deformity determination logic circuitry may request a user to orient images a certain way dependent upon the anatomy and view. For example, a left tibia should be oriented with proximal at the top of the screen, lateral to the left, distal to the bottom, and medial to the right. The mechanical axis may fine tune the coordinate system. The deformity determination logic circuitry may default the mechanical to vertical so if the image is oriented with proximal perfectly at the top of the screen and distal perfectly at the bottom then no further action is required. If the mechanical axis is not perfectly vertical then it can be adjusted. In the left tibia example, the mechanical axis may be set at a 45 degree angle. The deformity determination logic circuitry may define the top most end of the axis as proximal and define the bottom most end as distal. The deformity determination logic circuitry may define medial and lateral perpendicular to the mechanical axis to fully define the coordinate system.

Some embodiments automatically rotate the lines through the reduction points and the additional interconnection points about the concentric location of the first and second reduction points to make the lines colinear. Further embodiments may also receive graphical input and rotate one or both bone segments based on the graphical input to align the first bone segment and the second bone segment.

Some embodiments use 3D images, such a CT scans, MRI scans, etc., instead of two-dimensional (2D) images to reduce the two bone segments. With three-dimensional image modalities, the user must create three-dimensional planes rather than two-dimensional planes. A user may place three points or more on the first bone segment so that the deformity determination logic circuitry may generate a three-dimensional plane for the first bone segment. Three points may also be placed on the second bone segment so that the deformity determination logic circuitry may draw a second plane on the second bone segment. The points should be placed such that the planes of the first and the second bone segments are aligned (coplanar) when the bone segments are aligned. Algorithms may be used to place the points/planes automatically on each bone segment.

When using three-dimensional image modalities, many embodiments may align the plane generated by the points on the second bone segment with the plane generated by the points on the first bone segment to present a modified display of the bone segments. Some embodiments may also collocate points placed on the first bone segment with corresponding points placed on the second bone segment to further orient the second bone segment relative the first bone segment. Some embodiments may use algorithms to determine the best fit of the two cut surfaces. Some embodiments may require that the points be placed at specific anatomic locations in order to create coordinate systems for each bone segment which can be used for orienting the bone segments. Further embodiments record each translation and/or angulation of one or both bone segments. Other embodiments compare the positions of one or both bone segments at least once, such as after the alignment of the bone segments is approved, to determine each translation and angulation of the bone segments.

In some embodiments using three-dimensional imaging modalities, the alignment of one bone segment to the other bone segment plane may provide an estimate of the six deformity parameters. The anatomical directions necessary to orient the calculated translations and angulations may be derived from imaging markers within the image, user input, required orientation of the images, or hardware orientation.

Some embodiments perform the alignment of one bone segment plane to the other bone segment plane and other movements dictated by the points placed on the bone segments automatically after the user has placed all six points. Other embodiments receive graphical input and perform the translation and angulations based on or responsive to the graphical input. For instance, using three-dimensional imaging modalities, many embodiments may automatically calculate an axial angulation.

In some embodiments, the user may compare the axial angulation determined clinically against the axial angulation determined via the three-dimensional imaging modalities. Further embodiments may allow the user to add one or more additional axial angulations to the comparison. Such embodiments may generate and present images of the corrected bone segments based on the two or more different axial angulations. Further embodiments may present the images individually on a screen, side-by-side, and/or overlapping. In some embodiments, the user may move one of the corrected images to overlap one or more of the other corrected images to perform the comparison. The user may then select the axial angulation for calculation of the deformity parameters based on review of the alternative corrected images.

Several embodiments identify a fixed bone segment (a bone segment with a fixed position and rotation) such as via input from a user and only move the other bone segment. In many embodiments discussed herein, the first bone segment may be in a fixed position and rotation and the second bone segment may be moved to align the second bone segment with the first bone segment, but embodiments are not limited to such a relationship. For instance, some embodiments may facilitate translation and angulation of both bone segments or may fix the second bone segment and may translate and rotate the first bone segment.

Several embodiments may then receive graphical input via the modified image to make fine adjustments to the alignment of the bone segments represented in the modified image. For instance, a user may determine that the modified image does not illustrate a satisfactory alignment of the first and second bone segments, so such embodiments may adjust the alignment illustrated in the modified image based on graphical input from the user. Such embodiments may nudge or adjust the, e.g., AP or LAT translation, the axial translation, and/or the AP or LAT angulation illustrated in the modified image based on input from the user.

In a 3D reduction environment, adjusting the translation and angulation of the bone segments can be in an oblique plane (non-AP/LAT) that represents the maximum deformity plane image. Translation and rotation (nudging) of each segment can be made about independent three-dimensional coordinate systems.

In many embodiments, the axial angulation may be determined clinically, e.g., by physical examination of the corresponding patient. Other embodiments may provide a transverse plane image to receive graphical input for axial angulation. Further embodiments may determine the axial angulation by collocating points identified on a first bone segment with points identified on a second bone segment of a 3D image and measuring angulation required for collocation.

While many embodiments herein discuss an exterior fixator for tibia and fibula fractures, embodiments are applicable to deformations of any fractured or osteotomized bones. Furthermore, embodiments described herein focus primarily on a single fracture that separates a bone into two bone segments, but embodiments are not limited to a single fracture of, e.g., a tibia or fibula. Embodiments may address each pair of bone segments separately and the bone segments may be part of any bone. For instance, a tibia may be fractured or osteotomized into three bone segments, i.e., a first bone segment, a second bone segment, and a third bone segment. Such embodiments may identify the deformity of the first bone segment and the second bone segment and identify the deformity of the third bone segment with respect to the second bone segment.

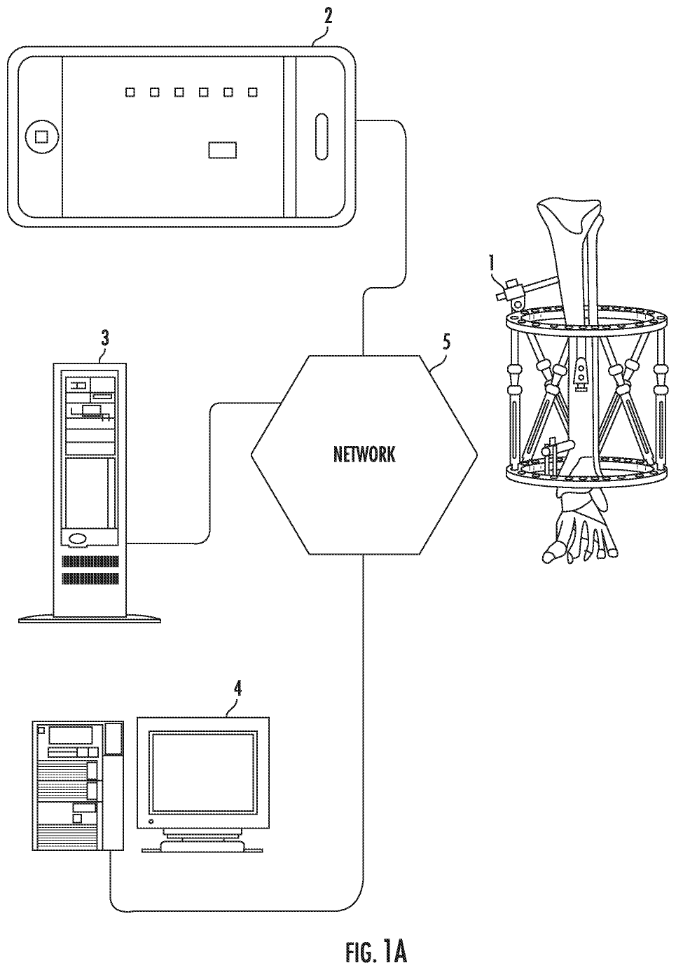

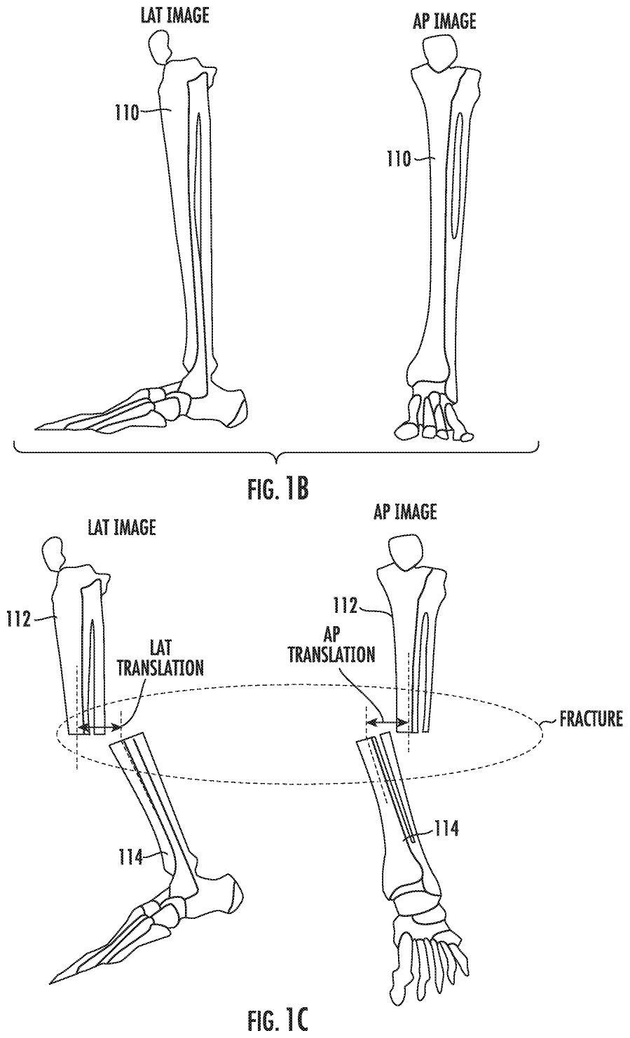

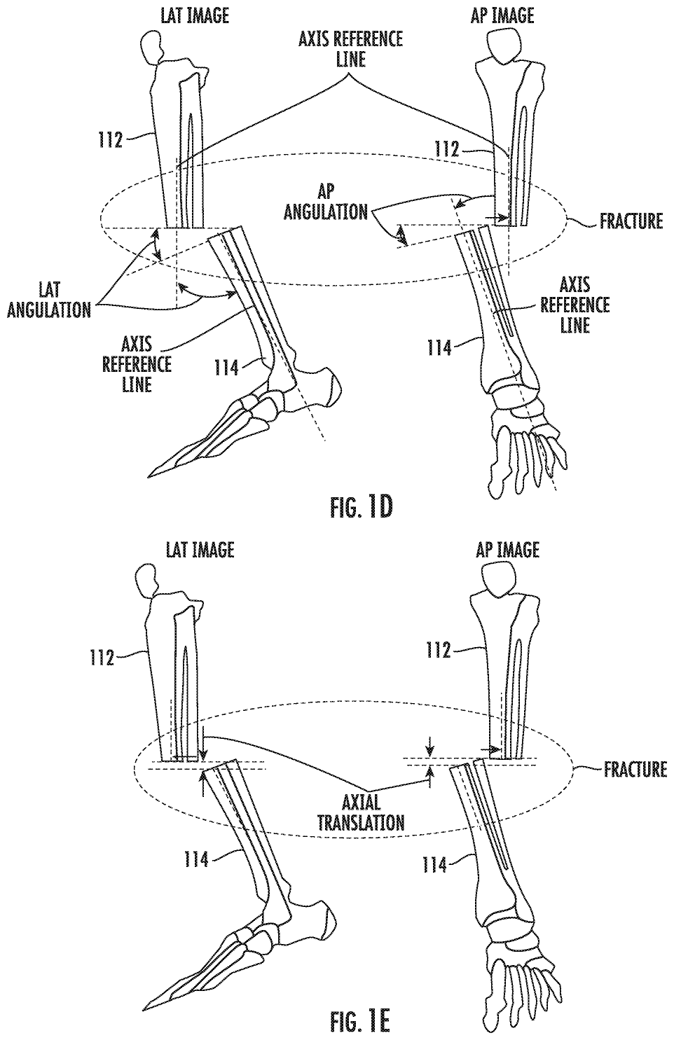

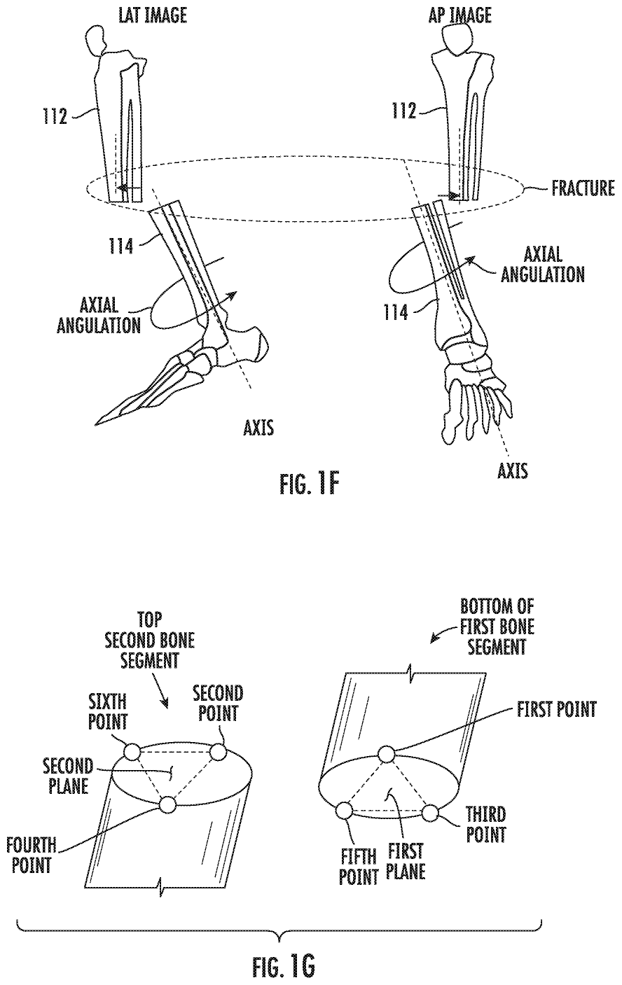

An embodiment of a system for treating a patient is illustrated in FIG. 1A. The system illustrated is only one example of a system and includes only one example of deformity analysis and/or correction planning discussed herein. Other systems may use the deformity parameters for other types of bone alignment devices, fractures, deformity correction, joint replacements/fusions, and/or for, e.g., navigated surgery such as a navigated surgery to install a bone alignment device such as the external bone alignment device 1.

The system may include the bone alignment device 1 configured to be coupled to a patient, a patient device 2 connected to a network 5, a server computer 3 connected to the network 5, and a Health Care Practitioner (HCP) device 4 connected to the network 5. The illustrated bone alignment device 1 may comprise a six-axis external fixator. In other embodiments, a bone alignment device 1 may be any device capable of coupling to two or more bones or pieces of bone and moving or aligning the bones or pieces of bone relative to one another. In yet other embodiments, a device for use in a system within the scope of embodiments may be any type of medical device for which a set of deformity parameters for two or more bone segments may be beneficial.

The patient device 2 illustrated is a handheld wireless device. In other embodiments, a patient device may be any brand or type of electronic device capable of executing a computer program and outputting results to a patient. For example, and without limitation, the patient device 2 may be a smartphone, a tablet, a mobile computer, or any other type of electronic device capable of providing one or both of input and output of information. In some embodiments, the patient device 2 may be a patient owned device. In some embodiments, the patient device 2 may be a handheld device or a desktop device. Such a device may provide ready access for input and output for a patient to whom a medical device such as the bone alignment device 1 is coupled. A patient device such as the patient device 2 may be distinguishable from an HCP device such as the HCP device 4 at least in that a patient device would not necessarily require permission or interaction from an HCP in order for a patient to transmit or receive information regarding the patient's treatment through the patient device 2.

A patient device such as the patient device 2 may be connected to the network 5 by any effective mechanism. For example, and without limitation, the connection may be a wired and/or wireless connection, or any combination thereof, through any number of routers and switches. Data may be transmitted by any effective data transmission protocol. Any patient device of the system may include integrated or separate computer readable media containing instructions to be executed by the patient device. For example, and without limitation, computer readable media may be any media integrated into the patient device such as a hard disc drive, random access memory (RAM), or non-volatile flash memory. Such computer readable media, once loaded into the patient device, may be integrated and non-transitory data storage media. Similarly, computer readable media may be generally separable from the patient device, such as a flash drive, external hard disc drive, Compact Disc (CD), or Digital Versatile Disc (DVD) that is readable directly by the patient device or in combination with a component connectable to the patient device.

The network 5 may be one or more interconnected networks, whether dedicated or distributed. Non-limiting examples include personal area networks (PANs), local area networks (LANs), wide area networks (WANs), metropolitan area networks (MANs), private and/or public intranets, the Internet, cellular data communications networks, switched telephonic networks or systems, and/or the like. Connections to the network 5 may be continuous or may be intermittent, only providing for a connection when requested by a sending or receiving client.

The server computer 3 is shown connected to the network 5 in FIG. 1. The server computer 3 may be a single computing device in some embodiments or may itself be a collection of two or more computing devices and/or two or more data storage devices that collectively function to process data as described herein. The server computer 3, or any one or more of its two or more computing devices, if applicable, may connect to the network 5 through one or both of firewall and web server software and may include one or more databases. If two or more computing devices or programs are used, the devices may interconnect through a back-end server application or may connect through separate connections to the network 5. The server computer 3 or any component server device of the system may include integrated or separate computer readable media containing instructions to be executed by the server computer. For example, and without limitation, computer readable media may be any volatile or non-volatile media integrated into the server computer 3 such as a hard disc drive, random access memory (RAM), or non-volatile flash memory. Such computer readable media, once loaded into the server computer 3 as defined herein, may be integrated, non-transitory data storage media. In some embodiments, a server computer 3 may include a storage location for information that will be eventually used by the patient device 2, the server computer 3, and/or the HCP device 4.

When stored on the server computer 3, memory devices of the server computer 3, as defined herein, provide non-transitory data storage and are computer readable media containing instructions. Similarly, computer readable media may be separable from the server computer 3, such as a flash drive, external hard disc drive, tape drive, Compact Disc (CD), or Digital Versatile Disc (DVD) that is readable directly by the server computer 3 or in combination with a component connectable to the server computer 3.

In some embodiments, deformity determination logic circuitry of the server computer 3 may communicate with the HCP device 4 via, e.g., a web browser or other client software installed on the HCP device 4 (deformity determination logic circuitry) to facilitate interaction with a user such as an orthopedic surgeon to describe a deformity based on a set of one or more images such as radiographs. The HCP device 4 may upload one or more images of the deformity via the network 5. In other embodiments, the deformity determination logic circuitry may reside on and may comprise, e.g., code for execution by a processor of the HCP device 4 so that a network may not be required.

The one or more images may be a single image such as a radiograph of a first and second bone segment for a two-dimensional description of the deformity and may be two 2D images or one 3D image for a three-dimensional description of the deformity. Additional medical imaging (e.g., magnetic resonance imaging (MRI), computed tomography (CT), x-ray, ultra-sound, etc.) can be used to create a 3D model of the patient's bone to analyze deformity parameters of the bone segments. In some embodiments, the one or more images may include additional images if the code is part of a more complex software application that offers functionality other than just analysis of a deformity. For instance, a hexapod software application may use deformity parameters from the deformity analysis and additional inputs to determine a strut adjustment schedule or prescription for the external bone alignment device 1. For example, the deformity determination logic circuitry may use one or more or any combination of edge and image recognition software, x-ray markers, manual inputs, automated inputs, augmented reality systems, and sensor technologies.

The software may display a 2D image with at least two bone segments in a first plane. The user may indicate, through graphical inputs, locations for interconnection points between the first and second bone segments such as a first reduction point, a second reduction point, and possibly one or more additional interconnection points in any order based on a preference of a user, in a default order, or in a predefined order established by the deformity determination logic circuitry. In other embodiments, the user may indicate, through graphical inputs, locations for two interconnection points on the first bone segment and two corresponding interconnection points on the second bone segment. The deformity determination logic circuitry may calculate the first reduction point on the first bone segment based on calculation of the midpoint between the two interconnection points on the first bone segment. The deformity determination logic circuitry may also calculate the second reduction point on the second bone segment based on calculation of the midpoint between the two interconnection points on the second bone segment.

For the purposes of the following discussions, the first and the third points may comprise interconnection points on the first bone segment to associate with the second and fourth points, respectively, which comprise interconnection points on the second bone segment. In some embodiments, the first interconnection point may comprise the first reduction point and the second interconnection point may comprise the second reduction point. In other embodiments, the first reduction point is the midpoint between the first and third interconnection points on the first bone segment and the second reduction point is the midpoint between the second and fourth interconnection points on the second bone segment. The first and second reduction points may represent translation points to be brought together and the resulting co-centric point may represent a pivot point for angulation of the bone segments to align the bone segments.

Embodiments utilizing a 3D image modality may require additional points. The first, third, and fifth points may comprise points on the first bone segment within a first plane to connect with second, fourth, and sixth points, respectively, within a second plane on the second bone segment. Some 3D embodiments may require that first and second, third and fourth, and fifth and sixth point pairs be placed on their associated bone segments such that if the two segments were properly aligned the first and second points would be collocated, the second and third points would be collocated, and the fifth and sixth points would be collated. Some embodiments that allow for 3D images may also treat the first and second reduction points as translation points and move the first and second reduction points to be concentric to create a pivot point.

The deformity determination logic circuitry of the software may overlay the first reduction point on a first bone segment and overlay the second reduction point on the second bone segment on the 2D image or section of the 3D image.

In many embodiments, the deformity determination logic circuitry of the software may overlay the third point on a first bone segment and overlay a first line interconnecting the first reduction point and the third point. Similarly, the deformity determination logic circuitry of the software may overlay the fourth point on a second bone segment and overlay a second line interconnecting the second reduction point and the fourth point. The generated lines may or may not be displayed to the user. Other embodiments for use with 2D images may allow the user to overlay a line running through the first reduction point and a line running through the second reduction point directly rather than by overlaying the third and fourth points on the 2D images.

For 3D images, some embodiments of the deformity determination logic circuitry of the software may overlay three points on a first bone segment and three points on a second bone segment as described. Lines between the points are unnecessary (but may optionally be shown) as three points in space may be used to generate a plane. The three points on each bone segment may be used to generate a plane on each bone segment as shown in FIG. 1G. Other embodiments using 3D image modalities may allow users to select a face of each bone segment and generate the planes normal to the selected faces rather than requiring three points be overlaid for each segment.

After determining and possibly overlaying one or more of the lines, the software may divide the image along the cut line (or cut plane for 3D images), bring the second reduction point and the associated image segment to the first reduction point, and, in some embodiments, generate a modified image illustrating the concentric reduction points. The cut line or cut plane may be defined, in some embodiments, by the interconnection points identified on the second bone segment and, in other embodiments, based on another line or by the interconnection points identified on the first bone segment. In several embodiments, the software may automatically, or through interaction from the user, align a second line through interconnection points on the second bone segment (or second plane on the second bone segment for 3D images) and the associated image segment with a first line through interconnection points on the first bone segment (or first plane on the first bone segment for 3D images) to cause the first line and the second line to be collinear (or to cause the first plane to be coplanar with the second plane for 3D images).

For 3D images, in some embodiments, the software may automatically, or through interaction of the user, collocate associated point pairs. In some 3D embodiments, the first and second reduction points may be collocated as a 3D pivot point of the bone segments. In other embodiments, multiple point pairs may be collocated. If the point pairs were overlaid on identical locations on the associated bone fragments, then the combination of collocating two points and making the two planes coplanar is sufficient to reduce a fracture in all six degrees of freedom. Some embodiments of the software may use edge detection algorithms to align the two bone segments after the planes of each bone segment such that the relative locations of the points on each bone segment is not critical.

After the software aligns the second line and the associated image segment with the first line (or aligns the first plane with the second plane to be coplanar and either collocates points or aligns the bone segments by other means for 3D images), the bone segments in the first and second portions of the image will be at least roughly aligned and a modified image illustrating the alignment can be displayed. In many embodiments, the deformity determination logic circuitry may provide an opportunity for a user to adjust the alignment if the user determines that the first and second bones segments are not well-aligned, or the alignment could otherwise be improved. In many such embodiments, the deformity determination logic circuitry may allow the user to overlay the one or more reference lines on the modified image. For instance, one reference line may comprise a straight line through the axis of the first bone segment and a second reference line may comprise a straight line through the axis of the second bone segment. When bone segments are properly aligned, the axes of the two fragments are collinear so some embodiments may provide only one axis line through one of the bone segments for the user to assess if the proper alignment has been achieved.

If unsatisfied with the alignment of the bone segments, the user may graphically adjust the position and/or orientation of the first and/or second bone segments by dragging the image segments to new positions and/or orientations until satisfied. Some embodiments include nudge tools (e.g. graphical buttons to add or subtract 1 millimeter (mm) of translation medially, add or subtract 2 degrees of valgus about the midpoint of the second line, add or subtract 1 millimeter (mm) of “short” translation vertically and/or the like) for changing the position and/or orientation of the bone segments. In some embodiments, the nudge tools may initially control angular corrections of the second bone segment about the concentric first and second reduction points. In further embodiments, the nudge tools may unlock the position of the rotation point (no longer limited to concentric first and second reduction points) to reposition one or both to different locations as needed.

In many embodiments, the deformity determination logic circuitry may record the movement of the image segments to determine deformity parameters for each image processed as discussed above. For example, after placement of the first and second reduction points, the deformity determination logic circuitry may record in memory, possibly in a data structure such as a vector or table, components of translation of the second reduction point to make the second reduction point concentric with the first reduction point. If the image is, e.g., a LAT radiograph with an established coordinate system in the software, the horizontal translation may represent a LAT View translation and the vertical translation may represent an axial translation. Vertical and horizontal references may assume that movement between the top and bottom of the radiograph are vertical movements and that movement from side to side of the radiograph are horizontal movements. Other labels can be used also such as up and down, left and right, medial, short, and/or the like. Note that vertical and horizontal movement may be relative to the axis of a selected one of the bone segments such as a bone segment selected to be a stationary bone segment for the purposes of determining relative adjustments or movements of other bone segments.

Note that embodiments can use images captured from any angle or orientation and movements of bone segments may be defined in relation to the coordinate system implemented by the deformity determination logic circuitry. Thus, references to vertical or horizontal movements relative to a 2D or 3D image may not reflect the actual components of such movements determined and stored by the deformity determination logic circuitry unless properly oriented by the user. For instance, a vertical movement with respect to a particular image may represent movement along an x-axis, a y-axis, a z-axis, or any combination thereof, with respect to the coordinate system implemented by the deformity determination logic circuitry. Thus, the deformity determination logic circuitry may record such movements as a tuple or vector such as (x, y, z), where x, y, and z represent numbers indicative of movement in units such as millimeters or centimeters along the x-axis, y-axis, and z-axis, respectively. A movement of zero, in some embodiments, may represent no movement, a negative movement may represent movement in a first direction with respect to an axis, and a positive movement may represent movement in a second direction with respect to the axis.

AP and LAT views are common practice for radiographs of fractures, but embodiments are not limited to AP and LAT view images. Furthermore, as long as each of the images has a known scale, the images do not have to be the same scale. The deformity determination logic circuitry may translate or convert scales to a selected or default scale implemented by the deformity determination logic circuitry and translate or convert movements associated with bone segments in images to a coordinate system implemented by the deformity determination logic circuitry.

Note that while some embodiments herein describe movements of one or both bone segments as vertical movement or horizontal movement, embodiments may not be so limited. For example, each movement of a bone segment may involve one or more different components of movement depending on the orientation of the images and the coordinate system established or chosen for the deformity determination logic circuitry. So a movement by a bone segment on an image to the left or right may involve one or more components of movement along an x-axis, a y-axis, and/or a z-axis of the coordinate system established for the deformity determination logic circuitry. The same is true for up and down movement of the bone segment.

In addition to recording the translations, the deformity determination logic circuitry may also record the rotation of the second image about the concentric reduction points to bring the first and second lines together. Note that if a cut line is implemented and the deformity determination logic circuitry interacts with the user to determine the rotation of the second image rather than bringing the first and the second lines together, the deformity determination logic circuitry may record the rotation graphically input by the user. For the LAT radiograph, the rotation may represent the LAT view angulation and, in many embodiments, the rotation may be recorded in units of degrees.

In some embodiments the software may record nudges made by the nudge tool. In such embodiments, the software may combine the movements for each deformity parameter to determine the set of deformity parameters. Further embodiments may compare the resulting positions of the first and second bone segments to the original positions of the first and second bone segments to determine the deformity parameters. In such further embodiments, the nudge tools may be an independent software package and may not be part of the deformity determination logic circuitry.

The software can calculate only two-dimensional deformity with two dimensional images. When calculating 3D deformity parameters, the software may require analysis of, and thus process, at least two scaled images of the bone segments captured at different angular orientations with a common point between the two images or a single 3D image file such as a CT scan, MRI scan, or other know 3D medical imaging modality. For instance, after determining the LAT translation, LAT angulation, and the axial translation from the LAT radiograph, the user must analyze an AP radiograph with the software to complete the deformity analysis. Some embodiments require that the reduction points of the AP and LAT radiographs be located at the same 3D location in the two images in order to correlate the deformity parameters measured from the two images.

Note that the software may record an axial translation related to both the LAT radiograph and the AP radiograph. Considering that the reduction points may reflect graphical inputs on two different radiographs with different orientations, the axial translation determined from the LAT radiograph may not exactly match the axial translation determined from the AP radiograph so this potential conflict may have to be addressed by the deformity determination logic circuitry. In some embodiments, the deformity determination logic circuitry may resolve the conflict by interaction with the user of the HCP device 4 and/or by additional information analyzed by the deformity determination logic circuitry or otherwise received by the deformity determination logic circuitry.

Note that embodiments are not limited to the deformity determination logic circuitry residing in the server computer 3. The deformity determination logic circuitry may reside in whole or in part in the HCP device 4. The deformity determination logic circuitry may reside in whole or in part in the server computer 3. Furthermore, the deformity determination logic circuitry may reside partially in multiple computer servers and data storage servers managed by a management device and operating as the server computer 3. The deformity determination logic circuitry may also or alternatively reside partially in multiple computers and/or storage devices such as the HCP device 4. Where the deformity determination logic circuitry may reside partially in multiple computers, the deformity determination logic circuitry may include management logic circuitry to manage multiple local and/or remote resources.

The HCP device 4 is shown connected to the network 5. The HCP device 4 illustrated is a desktop personal computer. In other embodiments, the HCP device 4 may be any brand or type of electronic device capable of executing a computer program and receiving inputs from or outputting information to a user. For example, and without limitation, the HCP device 4 may be a smartphone, a tablet computer, or any other type of electronic device capable of providing one or both of input and output of information. Such a device may provide an interface for data input, compliance monitoring, prescription modification, and communication with a patient, another HCP, or a device or system manufacturer. An HCP device such as the HCP device 4 may be connected to the network 5 by any effective mechanism. For example, and without limitation, the connection may be by wired and/or wireless connection through any number of routers and switches. Data may be transmitted by any effective data transmission protocol. The HCP device 4 may include integrated or separate computer readable media containing instructions to be executed by the HCP device 4. For example, and without limitation, computer readable media may be any media integrated into the HCP device 4 such as a hard disc drive, RAM, or non-volatile flash memory. Such computer readable media once loaded into the HCP device 4 as defined herein may be integrated and non-transitory data storage media. Similarly, computer readable media may be generally separable from the HCP device 4, such as a flash drive, external hard disc drive, CD, or DVD that is readable directly by the HCP device 4 or in combination with a component connectable to the HCP device 4.

FIGS. 1B-1F illustrate LAT and AP images of an unfractured tibia 110 and the same tibia fractured or osteotomized into a first bone segment 112 and a second bone segment 114. Each of FIGS. 1C-1F illustrate at least one of the deformity parameters on the LAT image and the AP image. Note that while the illustrations focus on the tibia and LAT and AP images, embodiments may process any other bone and any other viewing angle in a similar manner.

FIG. 1B illustrates an embodiment of a LAT image of an unfractured tibia 110. Note that the AP image provides a fontal view of the tibia and the LAT view provides a side view of the tibia.

FIG. 1C illustrates an embodiment of the tibia bone 110 fractured or osteotomized into two bone segments, a first bone segment 112 and a second bone segment 114. As discussed therein, the first bone segment typically refers to the fixed bone segment if the processing involves a fixed bone segment. For instance, some embodiments fix the first bone segment and all deformity parameters are determined based upon movement of the second bone segment to align the second bone segment with the first bone segment. Other embodiments may move and/or rotate either or both bone segments and may determine the deformity parameters by recording the movements of either or both bone segments and/or by comparison of the final positions of either or both bone segments against the original positions of either or both bone segments.

In FIG. 1C, the embodiment may determine the LAT translation based on a horizontal translation of the second bone segment 114 to align the second bone segment with the first bone segment 112 on the LAT image. Similarly, the embodiment may determine the AP translation based on a horizontal translation of the second bone segment 114 to align the second bone segment with the first bone segment 112 on the AP image. Other embodiments may determine the LAT or AP translation based on a horizontal translation of both the first bone segment and the second bone segment 114 to align the bone segments 112 and 114.

FIG. 1D illustrates an embodiment of the tibia bone 110 fractured or osteotomized into two bone segments, a first bone segment 112 and a second bone segment 114 for purpose of illustrating the deformity parameters of LAT angulation and AP angulation. The LAT angulation is the rotation of the second bone segment 114 required to align the first bone segment 112 with the second bone segment 114 on the LAT image. The AP angulation is the rotation of the second bone segment 114 required to align the first bone segment 112 with the second bone segment 114 on the AP image. As shown in FIG. 1D, an alternative way to illustrate and/or determine the LAT or AP angulation is to overlay a first axis reference line through the axis of the first bone segment 112, overlay a second axis reference line through the axis of the second bone segment 114, and measure the angle between the first and second axis reference lines. The angle between the first and second axis reference lines may be the LAT or AP angulation or an angulation suggested by the deformity determination logic circuitry, depending on which view is being measured.

FIG. 1E illustrates an embodiment of the tibia bone 110 fractured or osteotomized into two bone segments, a first bone segment 112 and a second bone segment 114 for purpose of illustrating the deformity parameter of axial translation. Many embodiments determine the axial translation as the vertical movement of either or both the first bone segment 112 and the second bone segment 114 to bring the two bone segments together. The initial estimate of the axial translation is based on the vertical movement to make the first reduction point and the second reduction point concentric. The initial estimate is based on graphical input from a user of the HCP device 4 in FIG. 1A such as an orthopedic surgeon. Many embodiments determine the final axial translation after offering the user an opportunity to adjust the alignment with, e.g., a nudge tool. For 2D deformity parameters, the final axial translation may be determined from a single image. For 3D deformity parameters, the final axial translation parameter may be determined after calculation of an axial translation for two or more images such as a LAT view and an AP view of the bone segments. In still other embodiments, a view may be selected for determining the axial translation prior to processing one or more images for deformity parameters and the deformity determination logic circuitry may only record movements related to and calculate and/or determine the axial translation based on the view selected for determining the axial translation.

FIG. 1F illustrates an embodiment of the tibia bone 110 fractured or osteotomized into two bone segments, a first bone segment 112 and a second bone segment 114 for purpose of illustrating the deformity parameter of axial angulation. The axial angulation is the rotation of the second bone segment 114 about the vertical axis of the second bone segment 114 to align the second bone segment with the first bone segment 112. In many embodiments, the axial angulation is determined clinically.

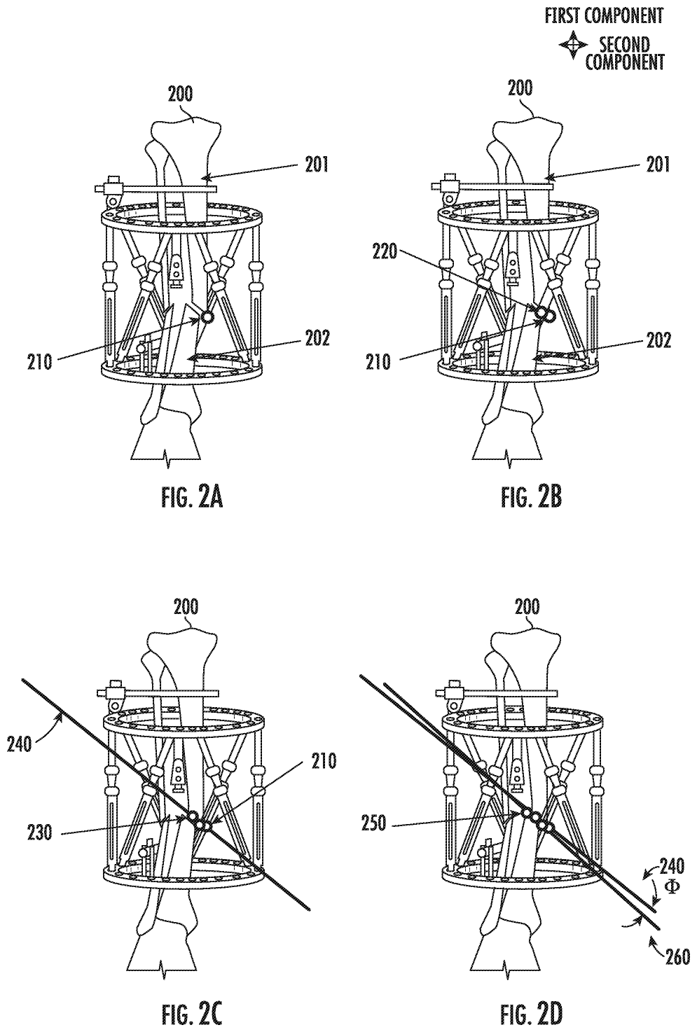

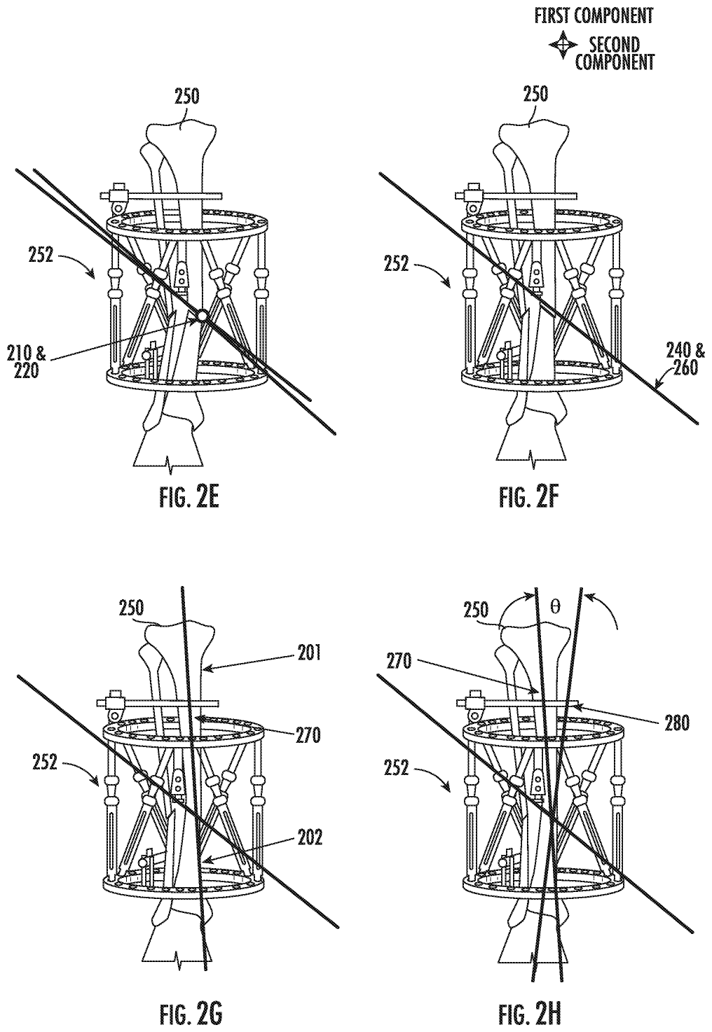

FIGS. 2A-I illustrate embodiments of modifications to a postoperative image of the same radiograph, or x-ray image, during a process of determining movements of two bone segments of a misaligned tibia to align the misaligned tibia by adjustment of the radiographs. The images may reside on the HCP device 3, the server computer 4, or both. Furthermore, the graphical manipulations of the images such as adding overlays can be created by deformity determination logic circuitry of the server computer 3 and/or the HCP device 4. In some embodiments, the deformity determination logic circuitry of the server computer 3 can instruct deformity determination logic circuitry of the HCP device to perform the graphical manipulations and, in other embodiments, the deformity determination logic circuitry of the server computer 3 can perform some of or all the graphical manipulations and transmit the modified images to the HCP device 4. In further embodiments, the deformity determination logic circuitry of the HCP device 4 can perform the graphical manipulations independently from the server computer 3 and report movements such as translations and rotations to the server computer 3. For example, the deformity determination logic circuitry may be the HCP device 4 comprising an HCP client software package that can perform a portion of the process or has tools to perform some of or all the manipulations of the images based on instructions from the deformity determination logic circuitry of the server computer 3.

Logic circuitry herein refers to a combination of hardware and code to perform functionality. For instance, the logic circuitry may include circuits such as processing circuits to execute instructions in the code, hardcoded logic, application specific integrated circuits (ASICs), processors, state machines, microcontrollers, and/or the like. The logic circuitry may also comprise memory circuits to store code and/or data, such as buffers, registers, random access memory modules, flash memory, and/or the like.

The deformity determination logic circuitry may reside entirely in the HCP device 4, partially in both the server computer 3 and the HCP device 4, or entirely in the server computer 3. For example, if the functionality is entirely in the server computer 3, the HCP device 4 may comprise a terminal with a display and one or more input devices such as a keyboard and mouse. The user may interact with the deformity determination logic circuitry in the server computer 3 via the display, keyboard and mouse.

If the functionality is entirely in the HCP device 4, the server computer 3 may act as storage for images, storage for code such as code to determine deformity parameters, and/or other data or code. In some embodiments, the server computer 3 may determine a user's access permissions to code and patient records, for instance, and may establish access to the data and transmit a code package from a storage medium (deformity determination logic circuitry) to the HCP device 4 for execution to determine deformity parameters.

In other embodiments, the server computer 3 may offer authentication services or may have no significant interaction with the HCP device 4 for the purpose of processing the first image to determine a deformity of the bone segments. For instance, the server computer 3 may provide authentication services to verify that a user has access to certain images, patient records, etc. In some embodiments, the server computer 3 may authenticate access to records, applications, and/or other resources stored locally at the HCP device 4 and/or stored remotely based on permissions associated with the user's credentials.

If the functionality of the deformity determination logic circuitry is partially in the server computer 3 and partially in the HCP device 4, the particular division of functionality may be based on the topology of the computer network, which can be complex in, e.g., hospitals. For example, the server computer 3 may assign compute resources and data storage resources for a specific task of determining the deformity parameters. In some embodiments, the server computer 3 may transmit a local code package for execution on an HCP device 4 located with the user and execute another code package on a compute server. In some embodiments, images may be transmitted to the HCP device 4 for processing. In other embodiments, the images may be accessed and processed by the server computer 3 and transmitted to the HCP device 4 to display to the user. Various embodiments may offer different distributions of functionality between the HCP device 4 and the server computer 3 for determining the deformity parameters.

FIG. 2A depicts an AP image of a right leg with an external fixator. The tibia and fibula are both fractured or osteotomized. This embodiment may determine the deformity parameters for the tibia.

The deformity determination logic circuitry may request, via the HCP device 4, that the user graphically select a location for a first reduction point 210 on the first bone segment 201. In response to a selection by the user, the image is modified as illustrated in FIG. 2A to include an overlay circle representing the first reduction point 210 at the location on the first bone segment 201 selected by the user. In further embodiments, the user may select the first and third interconnection points on the first bone segment 201 and the deformity determination logic circuitry may generate an overlay image of a circle at each interconnection point and calculate the first reduction point 210 as the midpoint between the first and third interconnection points (or as any other point relative to one or both of the first and third interconnection points). The deformity determination logic circuitry may include an overlay circle representing the first reduction point 210 at the midpoint between the first and third interconnection points on the first bone segment 201 (or at any other point relative to one or both of the first and third interconnection points). Note that while some of the embodiments require identification of the reduction points and/or interconnection points in a predefined order, some embodiments may receive such points in any order.

After selecting the first reduction point 210, the deformity determination logic circuitry may request that the user graphically select a second reduction point 220 on the second bone segment 202. The deformity determination logic circuitry may then generate an overlay image of a circle at the second reduction point 220 as illustrated in FIG. 2B. In further embodiments, the user may select the second and fourth interconnection points on the second bone segment 202 and the deformity determination logic circuitry may generate an overlay image of a circle at each interconnection point and calculate the second reduction point 220 as the midpoint between the second and fourth interconnection points (or as any other point relative to one or both of the second and fourth interconnection points). The deformity determination logic circuitry may include an overlay circle representing the second reduction point 220 at the midpoint between the second and fourth interconnection points on the second bone segment 202 (or at any other point relative to one or both of the second and fourth interconnection points).

FIG. 2C illustrates an overlay of a dot at a third point 230 on the first bone segment 201. Furthermore, upon selection of the third point 230 by the user, the deformity determination logic circuitry may generate a first line 240 that interconnects the first reduction point 210 and the third point 230 and overlay the image with the first line 240 as shown in FIG. 2C. In other embodiments, the deformity determination logic circuitry may define the first line 240 or generate an object that is the first line 240 but not illustrate the first line 240 on the image.

The user may graphically select a fourth point 250 on the second bone segment 202 and the deformity determination logic circuitry may create a second line 260 that interconnects the second reduction point 220 and the fourth point 250 and overlay the image with a representation of the second line. Some embodiments may also overlay an indication of the AP angulation phi, Φ, represented by the first line 240 and the second line 260 as illustrated in FIG. 2D. In other embodiments, the deformity determination logic circuitry may define the second line 260 or generate an object that is the second line 260 but not illustrate the second line 260 on the image.

Once the two reduction points are identified and the two additional points are identified, the present embodiment may generate a copy of the image, hide the portion of the image below the first line 240 on the original image 200 to create a first portion 250 with the original image 200, and hide the portion of the copied image above the second line 260 to create a second portion 252. If the location of the first portion is fixed, the deformity determination logic circuitry may collocate the second reduction point 220 with the first reduction point 210 as illustrated in FIG. 2E by moving the second reduction point 220 to the first reduction point 210, recording one component of the movement of the second portion 252 as an AP translation, and recording a second component of the movement of the second portion as an axial translation. In further embodiments, the deformity determination logic circuitry may define the first line 240 or the second line 260 as a cut line and hide, separate, or otherwise remove the portion of the image above the cut line to generate the second portion 252 of the image with the second bone segment 202.

After the present embodiment collocates the reduction points 210 and 220, the second portion 252 may be rotated automatically by the deformity determination logic circuitry or manually by a user via graphical input as shown in FIG. 2F. The second portion 252 may be rotated about the concentric reduction points 210 and 220 and, in many embodiments, the second portion 252 may be rotated until the first line 240 and the second line 260 are co-linear as illustrated in FIG. 2F.

Some embodiments of the deformity determination logic circuitry may generate or allow the user to automatically generate and overlay a reference line representing the axis of the first bone segment 201 as illustrated in FIG. 2G. Some of these embodiments may also automatically generate or allow the user to generate and overlay a reference line representing the axis of the second bone segment 202 as illustrated in FIG. 2H and some embodiments may also generate and overlay an indication of the rotation theta, θ, between the axis through the first bone segment and the axis through the second bone segment as illustrated in FIG. 2H.

With or without the reference lines, a user such as an orthopedic surgeon may determine if the first bone segment 201 and the second bone segment 202 are aligned. If the user determines that the bone segments 201 and 202 are not aligned well or the alignment can be improved, the user can change the alignment as shown in FIG. 2I via, e.g., nudge tools, or any other method either through graphical input or through keyboard input to rotate the second portion 252, translate the second portion 252, modify the location of the first reduction point 210 on the first bone segment 201, modify the location of the second reduction point 220 on the second bone segment 202, move the location of the rotation point, and/or the like. In some embodiments, the user may nudge the second bone segment 202 via graphical buttons and/or key strokes to add or subtract 1 or more (or a fraction of a) millimeter (mm) of translation medially, add or subtract 1 or more (or a fraction of a) degree of valgus about the midpoint of the second line, add or subtract 1 or more (or a fraction of a) millimeter (mm) of “short” translation vertically and/or the like.

Other embodiments may automatically rotate the second portion by theta, θ, based on the angular distance between the vertical axis through the first bone segment 201 and the vertical axis through the second bone second 202 to offer a possible correction to the user. In some embodiments, the user may determine to change the alignment as shown in FIG. 2I via, e.g., nudge tools, or any other method either through graphical input or through keyboard input to improve the alignment after accepting the proposed change automatically offered by the present embodiment.

All translations and rotations of the first portion 250 and the second portion 252 can be recorded and combined to determine the deformity parameters in some embodiments. In other embodiments, the final version of the image can be analyzed against the original image to determine the deformity parameters.

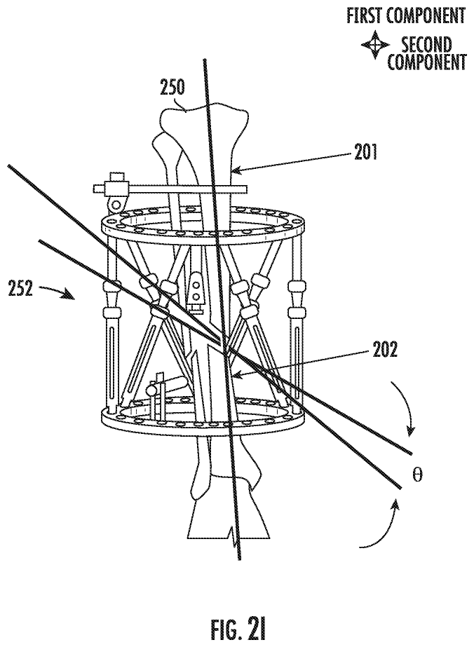

FIG. 3 depicts a flowchart 3000 of embodiments to identify movement of bone segments to align the bone segments. Flowchart 3000 may determine a set of deformity parameters related to two or more bone segments. The flowchart 3000 starts with identifying a first image to display, the first image including a first bone segment and a second bone segment (element 3010). For instance, a server computer such as the server computer 3 in FIG. 1A may comprise deformity determination logic circuitry to transmit or identify a scaled radiograph or other scaled image for a patient or to interact with a user of a computer such as the HCP device 4 in FIG. 1A to identify a scaled, first image for processing. In other embodiments, deformity determination logic circuitry of the HCP device may interact with a user to identify a scaled radiograph to process to determine deformity parameters Images can have any known scale or any scale that can be determined through analysis. Furthermore, the image may comprise a 2D image or a 3D image.

After identifying the first image, the remote computer may display the first image to facilitate graphical input and/or other input from a user of the remote computer. Thereafter, the user may identify a first reduction point on the first bone segment in the first image (element 3020) and identify a second reduction point on the second bone segment, the first and second reduction points to represent a connection point between the first and second bone segments (element 3030). The user may have one or more optional ways to identify the reduction point. For instance, the user may move a pointer with a mouse, trackball, keyboard, or other input device to a point on the first bone segment in the first image and, e.g., click the mouse button that the user considers to be an appropriate pivot point. In further embodiments, the user may identify the first reduction point by identifying two points on the first bone segment, a point such as the midpoint of the two points being the first reduction point. Similarly, the user may identify the second reduction point by identifying two points on the second bone segment, a point such as the midpoint of the two points being the second reduction point. In some embodiments, the two points on the first bone segment may represent interconnection points between the bone segments and the two points on the second bone segment may represent interconnection points between the bone segments.

In addition to identifying the first and second reduction points, the user may, in some embodiments, identify one or more additional points (element 3032) such as the two points on the first bone segment and the two points on the second bone segment. For instance, some embodiments may include an option to add one or more additional points and other embodiments may require one or more additional points. The user may identify a third point and a fifth point on the first bone segment and a fourth point and a sixth point on the second bone segment. The third, fourth, fifth, and sixth points should identify additional pairs of interconnection points on the bone segments that the user expects will connect when the bone segments are well aligned. In several embodiments, the fifth and sixth points are required when the first image is a 3D image to identify planes on the first and second bone segments. For instance, the first, third, and fifth points may identify a first plane on the first bone segment and the second, fourth, and sixth points may identify a second plane on the second bone segment as shown in FIG. 1G.

In some embodiments, a first line is automatically drawn by the deformity determination logic circuitry through the first reduction point and the third point in response to selection or identification of the third point by the user. Such embodiments may also automatically draw a second line through the second reduction point and the fourth point upon identification or selection of the fourth point by the user. Similar to the identification of the reduction points, the user may graphically select the third, fourth, fifth and sixth points on the first image via, input devices such as the mouse and/or keyboard.

In other embodiments, selection or identification of the third and fourth points may cause the deformity determination logic circuitry to create and overlay points on the image rather than lines. In some of these embodiments, the user may interact with the deformity determination logic circuitry to draw the first and second lines in addition to or instead of the third and fourth points.

In some embodiments, the deformity determination logic circuitry may generate one or more suggested reduction points and additional points. For instance, the deformity determination logic circuitry may analyze the first image to detect the edges of the bone segments automatically or via interaction with the user and identify one or more points along an edge of the bone segments either randomly, based on default or preferred criteria, or based on information related to selection of an ideal pivot point. The information related to selection of an ideal pivot point may be from the user or may be data provided to the deformity determination logic circuitry from another source.

Once the third and fourth points (and the fifth and sixth points or lines in some embodiments) are identified or selected, the deformity determination logic circuitry may, in some embodiments, copy the first image, hide the portion of the original image on the second bone segment side of first line or first plane (or where the first line or first plane can optionally be drawn), and hide the portion of the copied image on first bone segment side of second line or second plane (or where the second line or second plane can optionally be drawn) (element 3038). By this process, the first image is divided into two portions, the first portion including the first bone segment and the second portion including the second bone segment to allow movement of the portions to align the bone segments without substantial overlap. Hiding the portion may, in some embodiments, move that portion of the image or copied image to a hidden layer of the image and, in further embodiments, remove the hidden portion of the copied image or cover the hidden portion of the image with a solid background such as a black background on a layer below the images of the bone segments.

As an alternative to using the first and the second lines or planes to divide the first image into two portions, the user may, in some embodiments, divide the first image into a first portion with the first bone segment and a second portion with a second bone segment (element 3036) by any other means. For example, the user may interact with the deformity determination logic circuitry to draw a cut line or a cut plane between the two bone segments and the deformity determination logic circuitry may divide the image into two portions based on the cut line or cut plane. For example, to avoid modification of the original first image, the deformity determination logic circuitry may create a copy of the original image in memory or in a file in a storage device and the deformity determination logic circuitry may divide the image into two portions that can be moved independently.

In several embodiments, the deformity determination logic circuitry may create a cut line and overlay the cut line or plane (element 3039) on the first image to show the separation between the two portions. Depending on the embodiment, the HCP device 4 may divide the first image into two portions, the server computer 3 may divide the first image into two portions and transmit the two portions to the HCP device 4, or the server computer 3 may divide the first image into two portions and interact with the user via the HCP device 4 to facilitate movement of the portions via graphical input by the user.

After dividing the first image into a first portion and a second portion, the deformity determination logic circuitry may automatically or through interaction with a user, move the first portion and/or the second portion of the first image to collocate the first and second reduction points. The deformity determination logic circuitry may also communicate the modified first image to display the first bone segment connected to the second bone segment at the collocated first and second reduction points (element 3040). For instance, the server computer 3 may communicate the modified first image to the HCP device 4 and/or the HCP device 4 may communicate the modified first image to a graphics accelerator card, a graphics engine, and a graphical processor unit (GPU) to display the modified first image on a display.

After collocating the first reduction point and the second reduction point of the first portion and second portion of the first image, the deformity determination logic circuitry may interact with the user to optionally adjust the alignment of the first bone segment and the second bone segment (element 3045). For instance, if the first and second bone segments do not require rotation to align, the collocation of the reduction points may align the bone segments. On the other hand, if the bones segments require rotation for alignment, the deformity determination logic circuitry may interact with the user to rotate the second bone segment via rotation of the second portion of the first image about the concentric reduction points. The rotation may comprise an AP angulation, a LAT angulation, an axial angulation, another angulation, and/or the like, depending on the view provided by the first image and the orientation of the bone segments.

In some embodiments, the deformity determination logic circuitry may automatically rotate the second portion or suggest to the user a rotation of the second portion about the concentric reduction points to make the first line and the second line co-linear, or to make the first plane and the second plane coplanar for 3D images. For situations in which the user does not enter a third, fourth, fifth, and sixth point, the deformity determination logic circuitry may select one or more prospective rotations and suggest or illustrate the prospective rotations to the user. In a further embodiment, the user may provide graphical input or other input to indicate the magnitude of the rotation of the second portion about the concentric reduction points.

In other embodiments, the user may adjust one or more translations and/or rotations to align the bones segments in the first portion and the second portion of the modified first image. In a further embodiment, the user may enter prospective deformity parameters to determine well-aligned bone segments. In still another embodiment, the deformity determination logic circuitry may present an array of prospective adjustments in the graphical form by generation of an array of modified images for the user to review to facilitate selection of one of the prospective adjustments.

If unsatisfied with the alignment, the user may adjust the position of the moving segment relative to the reference (fixed) segment by any means. Adjustments to the position of the moving segment could include rotation or decoupling of the reduction points to allow for translation. The rotation point could be placed anywhere along the cut line (or cut plane) or the cut line (or cut plane) could be repositioned to allow full freedom with the rotation point. In many embodiments, the initial point of rotation could be a calculated or placed point other than one of the first reduction point, the second reduction point, the third point, the fourth point, the fifth point, and the sixth point discussed above.

After the user determines that the bone segments are well-aligned, the user may provide and the deformity determination logic circuitry may receive an indication representing approval of the modified first image for generation of deformity parameters (element 3060). For instance, the user may select a save function via a graphical input or a keyboard input to approve the modified image. If the desired deformity parameters are three-dimensional and the first image is not a 3D image, the deformity determination logic circuitry may determine another image should be processed (element 3070) and may determine to repeat elements 3010 through 3070 (element 3080).

On the other hand, if the desired deformity parameters are two-dimensional, if two images have already been processed, or if the first image is a 3D image, the deformity determination logic circuitry may determine not to process another image (element 3070). If more than one image for the same bone segments have been processed to determine the deformity parameters, the deformity determination logic circuitry may determine a common point between the more than one image and optionally resolve conflicts (element 3090). The images such as the first image can be scaled by any means. Thus, to determine and combine a set of deformity parameters from more than one image, the deformity determination logic circuitry requires a way to scale translations. In some embodiments, the deformity determination logic circuitry may receive manual inputs/measurements and/or perform automated scaling through recognizable objects of known size and shape. In other embodiments, the deformity determination logic circuitry may use a common point between the two images.

In some embodiments, when the first image is a 3D image or multiple 2D images have been processed by the deformity determination logic circuitry, the deformity determination logic circuitry may process additional 2D or 3D images to refine the measurements for determination of the deformity parameters or to refine the deformity parameters. For instance, multiple sets of measurements or deformity parameters can be combined in one or more different ways to derive a final set of measurements or deformity parameters. For example, the deformity determination logic circuitry may weight, average, determine a mean, and/or the like of individual measurements or deformity parameters or of sets of measurements or deformity parameters. Furthermore, the deformity determination logic circuitry may, in some embodiments, discard or reduce a weight associated with outliers of individual measurements or deformity parameters or with sets of measurements or deformity parameters when combining the measurements or deformity parameters.

The deformity determination logic circuitry may identify or calculate the common point between the scaled images from marker(s) or hardware component(s) visible in both images. The marker(s) or hardware component(s) may be of known (or measurable) size and shape at a known or specifiable location relative to the images that can be automatically or manually detected. Furthermore, the deformity determination logic circuitry may identify or calculate the common point between the scaled images from software input(s) placed by the user (likely but not limited to a graphically placed point on an easily recognizable anatomical landmark common between the two images). The choice of the common parameter between the two images is largely dependent on the application. In some embodiments, the choice could be made during the analysis or prior to the analysis as a user preference.

Conflict may involve differences in one or more deformity parameters determined from different images. One potential conflict may be the axial translation, which may be determined in many different angular orientations of the views of images for the bone segments. To resolve this conflict, the deformity determination logic circuitry may interact with the user to determine which axial translation should be used, or the deformity determination logic circuitry may select an axial translation based on other data, preferences, and/or the like,

Once the one or more images are processed for determination of the deformity parameters, the deformity determination logic circuitry may determine the deformity parameters by summing recorded movements of one or both bone segments or by comparing the original positions of the bone segments against the approved, aligned positions of the bone segments. For instance, the deformity determination logic circuitry may record each movement including rotations and translations of each bone segment so analysis of the movements can provide the deformity parameters. For embodiments in which one bone segment is considered to be fixed, the deformity determination logic circuitry may record only the movements and rotations of the other bone segment.

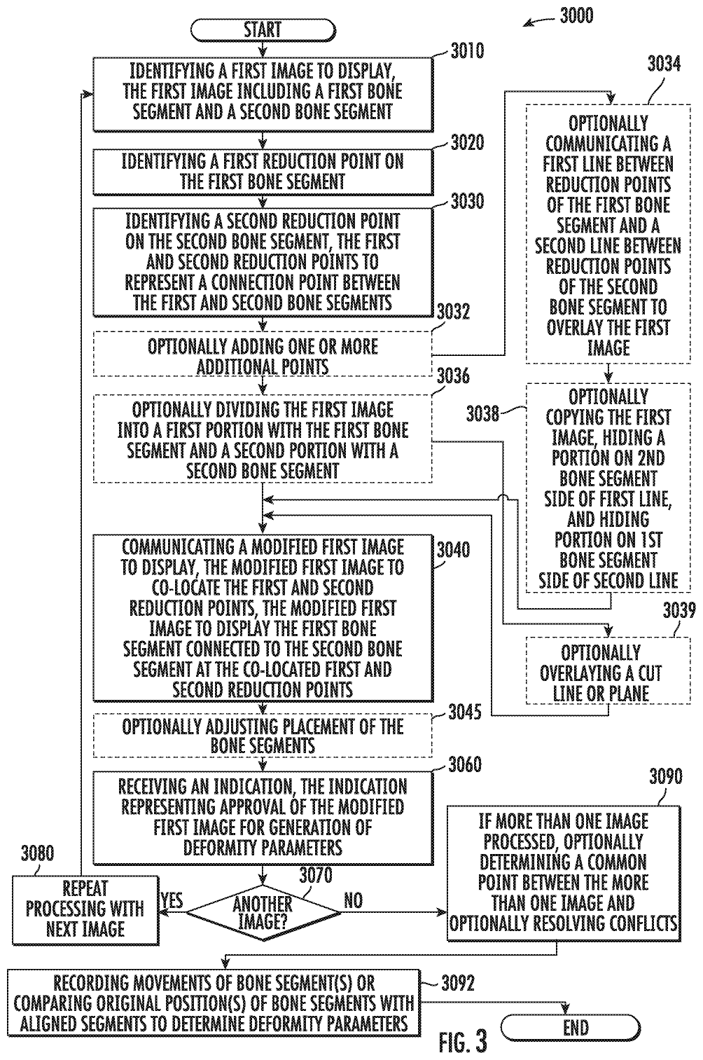

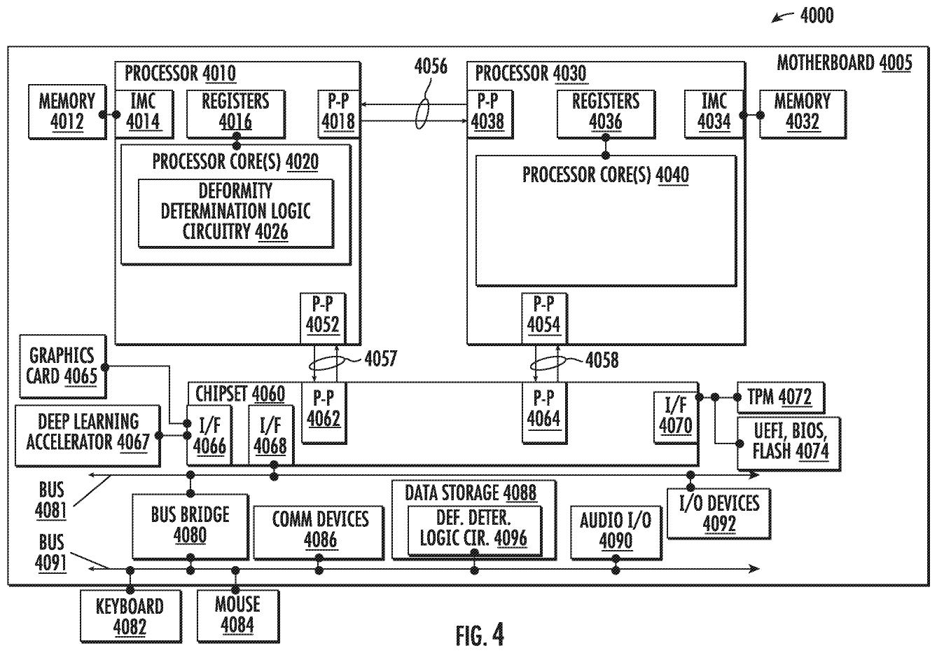

FIG. 4 illustrates an embodiment of a system 4000 such as the patient device 2, the server computer 3, and the HCP device 4 shown in FIG. 1A. The system 4000 is a computer system with multiple processor cores such as a distributed computing system, supercomputer, high-performance computing system, computing cluster, mainframe computer, mini-computer, client-server system, personal computer (PC), workstation, server, portable computer, laptop computer, tablet computer, handheld device such as a personal digital assistant (PDA), or other device for processing, displaying, or transmitting information. Similar embodiments may comprise, e.g., entertainment devices such as a portable music player or a portable video player, a smart phone or other cellular phone, a telephone, a digital video camera, a digital still camera, an external storage device, or the like. Further embodiments implement larger scale server configurations. In other embodiments, the system 4000 may have a single processor with one core or more than one processor. Note that the term “processor” refers to a processor with a single core or a processor package with multiple processor cores.

As shown in FIG. 4, system 4000 comprises a motherboard 4005 for mounting platform components. The motherboard 4005 is a point-to-point interconnect platform that includes a first processor 4010 and a second processor 4030 coupled via a point-to-point interconnect 4056 such as an Ultra Path Interconnect (UPI). In other embodiments, the system 4000 may be of another bus architecture, such as a multi-drop bus. Furthermore, each of processors 4010 and 4030 may be processor packages with multiple processor cores including processor core(s) 4020 and 4040, respectively. While the system 4000 is an example of a two-socket (2S) platform, other embodiments may include more than two sockets or one socket. For example, some embodiments may include a four-socket (4S) platform or an eight-socket (8S) platform. Each socket is a mount for a processor and may have a socket identifier. Note that the term platform refers to the motherboard with certain components mounted such as the processors 4010 and the chipset 4060. Some platforms may include additional components and some platforms may only include sockets to mount the processors and/or the chipset.

The first processor 4010 includes an integrated memory controller (IMC) 4014 and point-to-point (P-P) interconnects 4018 and 4052. Similarly, the second processor 4030 includes an IMC 4034 and P-P interconnects 4038 and 4054. The IMC's 4014 and 4034 couple the processors 4010 and 4030, respectively, to respective memories, a memory 4012 and a memory 4032. The memories 4012 and 4032 may be portions of the main memory (e.g., a dynamic random-access memory (DRAM)) for the platform such as double data rate type 3 (DDR3) or type 4 (DDR4) synchronous DRAM (SDRAM). In the present embodiment, the memories 4012 and 4032 locally attach to the respective processors 4010 and 4030. In other embodiments, the main memory may couple with the processors via a bus and shared memory hub.

The processors 4010 and 4030 comprise caches coupled with each of the processor core(s) 4020 and 4040, respectively. In the present embodiment, the processor core(s) 4020 of the processor 4010 include a deformity determination logic circuitry 4026 such as the deformity determination logic circuitry discussed in conjunction with FIG. 1A. The deformity determination logic circuitry 4026 may represent circuitry configured to implement the functionality of deformity determinations for bone segments in one or more images within the processor core(s) 4020 or may represent a combination of the circuitry within a processor and a medium to store all or part of the functionality of the deformity determination logic circuitry 4026 in memory such as cache, the memory 4012, buffers, registers, and/or the like. In several embodiments, the functionality of the deformity determination logic circuitry 4026 resides in whole or in part as code in a memory such as the deformity determination logic circuitry 4096 in the data storage unit 4088 attached to the processor 4010 via a chipset 4060 such as the deformity determination logic circuitry 1125 shown in FIG. 1B. The functionality of the deformity determination logic circuitry 4026 may also reside in whole or in part in memory such as the memory 4012 and/or a cache of the processor. Furthermore, the functionality of the deformity determination logic circuitry 4026 may also reside in whole or in part as circuitry within the processor 4010 and may perform operations, e.g., within registers or buffers such as the registers 4016 within the processor 4010, or within an instruction pipeline of the processor 4010.

In other embodiments, more than one of the processors 4010 and 4030 may comprise functionality of the deformity determination logic circuitry 4026 such as the processor 4030 and/or the processor within the deep learning accelerator 4067 coupled with the chipset 4060 via an interface (I/F) 4066. The I/F 4066 may be, for example, a Peripheral Component Interconnect-enhanced (PCI-e).

The first processor 4010 couples to a chipset 4060 via P-P interconnects 4052 and 4062 and the second processor 4030 couples to a chipset 4060 via P-P interconnects 4054 and 4064. Direct Media Interfaces (DMIs) 4057 and 4058 may couple the P-P interconnects 4052 and 4062 and the P-P interconnects 4054 and 4064, respectively. The DMI may be a high-speed interconnect that facilitates, e.g., eight Giga Transfers per second (GT/s) such as DMI 3.0. In other embodiments, the processors 4010 and 4030 may interconnect via a bus.

The chipset 4060 may comprise a controller hub such as a platform controller hub (PCH). The chipset 4060 may include a system clock to perform clocking functions and include interfaces for an input/output (I/O) bus such as a universal serial bus (USB), peripheral component interconnects (PCIs), serial peripheral interconnects (SPIs), integrated interconnects (I2Cs), and the like, to facilitate connection of peripheral devices on the platform. In other embodiments, the chipset 4060 may comprise more than one controller hub such as a chipset with a memory controller hub, a graphics controller hub, and an I/O controller hub.

In the present embodiment, the chipset 4060 couples with a trusted platform module (TPM) 4072 and the unified extensible firmware interface (UEFI), BIOS, Flash component 4074 via an interface (I/F) 4070. The TPM 4072 is a dedicated microcontroller designed to secure hardware by integrating cryptographic keys into devices. The UEFI, BIOS, Flash component 4074 may provide pre-boot code.

Furthermore, chipset 4060 includes an I/F 4066 to couple chipset 4060 with a high-performance graphics engine, graphics card 4065. In other embodiments, the system 4000 may include a flexible display interface (FDI) between the processors 4010 and 4030 and the chipset 4060. The FDI interconnects a graphics processor core in a processor with the chipset 4060.

Various I/O devices 4092 couple to the bus 4081, along with a bus bridge 4080 which couples the bus 4081 to a second bus 4091 and an I/F 4068 that connects the bus 4081 with the chipset 4060. In one embodiment, the second bus 4091 may be a low pin count (LPC) bus. Various devices may couple to the second bus 4091 including, for example, a keyboard 4082, a mouse 4084, communication devices 4086 and a data storage unit 4088 that may store code such as the deformity determination logic circuitry 4096. Furthermore, an audio I/O 4090 may couple to second bus 4091. Many of the I/O devices 4092, communication devices 4086, and the data storage unit 4088 may reside on the motherboard 4005 while the keyboard 4082 and the mouse 4084 may be add-on peripherals. In other embodiments, some or all the I/O devices 4092, communication devices 4086, and the data storage unit 4088 are add-on peripherals and do not reside on the motherboard 4005.

FIG. 5 illustrates an example of a storage medium 5000 to store code for execution by processors such as the deformity determination logic circuitry 4096 shown in FIG. 4. Storage medium 5000 may comprise an article of manufacture. In some examples, storage medium 5000 may include any non-transitory computer readable medium or machine readable medium, such as an optical, magnetic or semiconductor storage. Storage medium 5000 may store various types of computer executable instructions, such as instructions to implement logic flows and/or techniques described herein. Examples of a computer readable or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions may include any suitable type of code, such as source code, compiled code, interpreted code, executable code, static code, dynamic code, object-oriented code, visual code, and the like. The examples are not limited in this context.

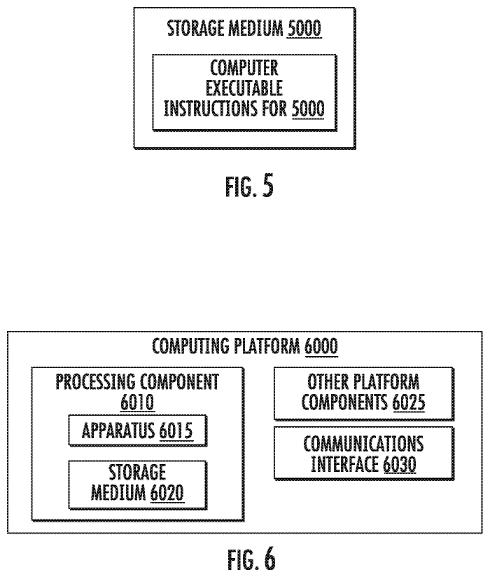

FIG. 6 illustrates an example computing platform 6000 such as the system 4000. In some examples, as shown in FIG. 6, computing platform 6000 may include a processing component 6010, other platform components or a communications interface 6030. According to some examples, computing platform 6000 may be implemented in a computing device such as a server in a system such as a data center or server farm that supports a manager or controller for managing configurable computing resources. Furthermore, the communications interface 6030 may comprise a wake-up radio (WUR) and may can wake up a main radio of the computing platform 6000.

According to some examples, processing component 6010 may execute processing operations or logic for apparatus 6015 described herein such as the deformity determination logic circuitry discussed in conjunction with FIGS. 1A and 4. Processing component 6010 may include various hardware elements, software elements, or a combination of both. Examples of hardware elements may include devices, logic devices, components, processors, microprocessors, circuits, processor circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements, which may reside in the storage medium 6020, may include software components, programs, applications, computer programs, application programs, device drivers, system programs, software development programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an example is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given example.

In some examples, other platform components 6025 may include common computing elements, such as one or more processors, multi-core processors, co-processors, memory units, chipsets, controllers, peripherals, interfaces, oscillators, timing devices, video cards, audio cards, multimedia input/output (I/O) components (e.g., digital displays), power supplies, and so forth. Examples of memory units may include without limitation various types of computer readable and machine readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory), solid state drives (SSD) and any other type of storage media suitable for storing information.

In some examples, communications interface 6030 may include logic and/or features to support a communication interface. For these examples, communications interface 6030 may include one or more communication interfaces that operate according to various communication protocols or standards to communicate over direct or network communication links. Direct communications may occur via use of communication protocols or standards described in one or more industry standards (including progenies and variants) such as those associated with the PCI Express specification. Network communications may occur via use of communication protocols or standards such as those described in one or more Ethernet standards promulgated by the Institute of Electrical and Electronics Engineers (IEEE). For example, one such Ethernet standard may include IEEE 802.3-2012, Carrier sense Multiple access with Collision Detection (CSMA/CD) Access Method and Physical Layer Specifications, Published in December 2012 (hereinafter “IEEE 802.3”). Network communication may also occur according to one or more OpenFlow specifications such as the OpenFlow Hardware Abstraction API Specification. Network communications may also occur according to Infiniband Architecture Specification, Volume 1, Release 1.3, published in March 2015 (“the Infiniband Architecture specification”).

Computing platform 6000 may be part of a computing device that may be, for example, a server, a server array or server farm, a web server, a network server, an Internet server, a work station, a mini-computer, a main frame computer, a supercomputer, a network appliance, a web appliance, a distributed computing system, multiprocessor systems, processor-based systems, or combination thereof. Accordingly, functions and/or specific configurations of computing platform 6000 described herein, may be included or omitted in various embodiments of computing platform 6000, as suitably desired.

The components and features of computing platform 6000 may be implemented using any combination of discrete circuitry, ASICs, logic gates and/or single chip architectures. Further, the features of computing platform 6000 may be implemented using microcontrollers, programmable logic arrays and/or microprocessors or any combination of the foregoing where suitably appropriate. It is noted that hardware, firmware and/or software elements may be collectively or individually referred to herein as “logic”.

It should be appreciated that the exemplary computing platform 6000 shown in the block diagram of FIG. 6 may represent one functionally descriptive example of many potential implementations. Accordingly, division, omission or inclusion of block functions depicted in the accompanying figures does not infer that the hardware components, circuits, software and/or elements for implementing these functions would necessarily be divided, omitted, or included in embodiments.

One or more aspects of at least one example may be implemented by representative instructions stored on at least one machine-readable medium which represents various logic within the processor, which when read by a machine, computing device or system causes the machine, computing device or system to fabricate logic to perform the techniques described herein. Such representations, known as “IP cores”, may be stored on a tangible, machine readable medium and supplied to various customers or manufacturing facilities to load into the fabrication machines that actually make the logic or processor.

Various examples may be implemented using hardware elements, software elements, or a combination of both. In some examples, hardware elements may include devices, components, processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. In some examples, software elements may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an example is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given implementation.

Some examples may include an article of manufacture or at least one computer-readable medium. A computer-readable medium may include a non-transitory storage medium to store logic. In some examples, the non-transitory storage medium may include one or more types of computer-readable storage media capable of storing electronic data, including volatile memory or non-volatile memory, removable or non-removable memory, erasable or non-erasable memory, writeable or re-writeable memory, and so forth. In some examples, the logic may include various software elements, such as software components, programs, applications, computer programs, application programs, system programs, machine programs, operating system software, middleware, firmware, software modules, routines, subroutines, functions, methods, procedures, software interfaces, API, instruction sets, computing code, computer code, code segments, computer code segments, words, values, symbols, or any combination thereof.

According to some examples, a computer-readable medium may include a non-transitory storage medium to store or maintain instructions that when executed by a machine, computing device or system, cause the machine, computing device or system to perform methods and/or operations in accordance with the described examples. The instructions may include any suitable type of code, such as source code, compiled code, interpreted code, executable code, static code, dynamic code, and the like. The instructions may be implemented according to a predefined computer language, manner or syntax, for instructing a machine, computing device or system to perform a certain function. The instructions may be implemented using any suitable high-level, low-level, object-oriented, visual, compiled and/or interpreted programming language.

Some examples may be described using the expression “in one example” or “an example” along with their derivatives. These terms mean that a particular feature, structure, or characteristic described in connection with the example is included in at least one example. The appearances of the phrase “in one example” in various places in the specification are not necessarily all referring to the same example.

Some examples may be described using the expression “coupled” and “connected” along with their derivatives. These terms are not necessarily intended as synonyms for each other. For example, descriptions using the terms “connected” and/or “coupled” may indicate that two or more elements are in direct physical or electrical contact with each other. The term “coupled,” however, may also mean that two or more elements are not in direct contact with each other, but yet still co-operate or interact with each other.

### Further Example Embodiments

Below are additional examples of embodiments:

In one embodiment, Example 1, a method to determine deformity parameters is disclosed. The method comprises: displaying a first image of a first bone segment and a second bone segment; identifying a first reduction point on the first bone segment and a second reduction point on the second bone segment, the first and second reduction points to represent a connection point between the first and second bone segments; displaying a modified first image, the modified first image to collocate the first and second reduction points, the modified first image to display the first bone segment connected to the second bone segment at the collocated first and second reduction points; and receiving an indication, the indication representing approval of the modified first image to determine deformity parameters.

In Example 2, the method of Example 1, further comprising: displaying a second image of the first bone segment and the second bone segment, the second image illustrating the first bone segment and the second bone segment at a perspective that is different from a perspective of the first image; identifying a third reduction point on the first bone segment; identifying a fourth reduction point on the second bone segment, the third and fourth reduction points to represent a second connection point between the third and fourth bone segments; displaying a modified second image, the modified second image to collocate the third and fourth reduction points, the modified second image to display the first bone segment connected to the second bone segment at the collocated third and fourth reduction points; and receiving an indication, the indication representing approval of the modified second image to determine the deformity parameters.

In Example 3, the method of Example 2, further comprising resolving a conflict between a deformity parameter common to the modified first image and the modified second image.

In Example 4, the method of Example 3, wherein the deformity parameter common to the modified first image and the modified second image comprises an axial translation.

In Example 5, the method of Example 1, further comprising: identifying a third point on the first bone segment; identifying a fourth point on the second bone segment, the third and fourth points to represent a second interconnection point between the first and second bone segments on the first image; and determining a cut line between the first bone segment and the second bone segment based on a line between the second reduction point and the fourth point.

In Example 6, the method of Example 5, the modified first image to display a first line between the first reduction point and the third point on the first bone segment.

In Example 7, the method of Example 6, the modified first image to display a second line between the second reduction point and the fourth point on the second bone segment.

In Example 8, the method of Example 1, wherein identifying a first reduction point on the first bone segment and a second reduction point on the second bone segment comprises identifying two points on the first bone segment, calculating the midpoint between the two points on the first bone segment, identifying two points on the second bone segment, and calculating the midpoint between the two points on the second bone segment, wherein the midpoint between the two points on the first bone segment is the first reduction point and the midpoint between the two points on the second bone segment is the second reduction point.

In Example 9, the method of Example 1, wherein the first bone segment and the second bone segment comprise two segments of a fractured bone.

In Example 10, the method of Example 9, further comprising identification of a cut line to separate the first image into a first portion comprising the first bone segment and a second portion comprising the second bone segment.

In Example 11, the method of Example 10, further comprising adjusting a position of the second portion of the first image to collocate the first reduction point and the second reduction point, to generate the first modified image.

In Example 12, the method of Example 1, further comprising adjusting a position of a copy of the first image to adjust alignment of the bone segments to create the modified first image.

In one embodiment, Example 13, an apparatus to determine deformity parameters is disclosed. The apparatus comprises: a means for displaying a first image of a first bone segment and a second bone segment; a means for identifying a first reduction point on the first bone segment and a second reduction point on the second bone segment, the first and second reduction points to represent a connection point between the first and second bone segments; a means for displaying a modified first image, the modified first image to collocate the first and second reduction points, the modified first image to display the first bone segment connected to the second bone segment at the collocated first and second reduction points; and a means for receiving an indication, the indication representing approval of the modified first image to determine deformity parameters.

In Example 14, the apparatus of Example 13, further comprising: a means for displaying a second image of the first bone segment and the second bone segment, the second image illustrating the first bone segment and the second bone segment at a perspective that is different from a perspective of the first image; a means for identifying a third reduction point on the first bone segment; a means for identifying a fourth reduction point on the second bone segment, the third and fourth reduction points to represent a second connection point between the third and fourth bone segments; a means for displaying a modified second image, the modified second image to collocate the third and fourth reduction points, the modified second image to display the first bone segment connected to the second bone segment at the collocated third and fourth reduction points; and a means for receiving an indication, the indication representing approval of the modified second image to determine the deformity parameters.

In Example 15, the apparatus of Example 14, further comprising a means for resolving a conflict between a deformity parameter common to the modified first image and the modified second image.

In Example 16, the apparatus of Example 15, wherein the deformity parameter common to the modified first image and the modified second image comprises an axial translation.

In Example 17, the apparatus of Example 13, further comprising: a means for identifying a third point on the first bone segment; a means for identifying a fourth point on the second bone segment, the third and fourth points to represent a second interconnection point between the first and second bone segments on the first image; and a means for determining a cut line between the first bone segment and the second bone segment based on a line between the second reduction point and the fourth point.

In Example 18, the apparatus of Example 17, the modified first image to display a first line between the first reduction point and the third point on the first bone segment.

In Example 19, the apparatus of Example 18, the modified first image to display a second line between the second reduction point and the fourth point on the second bone segment.

In Example 20, the apparatus of Example 13, wherein the means for identifying a first reduction point on the first bone segment and a second reduction point on the second bone segment comprises a means for identifying two points on the first bone segment, a means for calculating the midpoint between the two points on the first bone segment, a means for identifying two points on the second bone segment, and a means for calculating the midpoint between the two points on the second bone segment, wherein the midpoint between the two points on the first bone segment is the first reduction point and the midpoint between the two points on the second bone segment is the second reduction point.

In Example 21, the apparatus of Example 13, wherein the first bone segment and the second bone segment comprise two segments of a fractured bone.

In Example 22, the apparatus of Example 21, further comprising a means for identifying a cut line to separate the first image into a first portion comprising the first bone segment and a second portion comprising the second bone segment.

In Example 23, the apparatus of Example 22, further comprising a means for adjusting a position of the second portion of the first image to collocate the first reduction point and the second reduction point, to generate the first modified image.

In Example 24, the apparatus of Example 13, further comprising a means for adjusting a position of a copy of the first image to adjust alignment of the bone segments to create the modified first image.

In one embodiment, Example 25, a computer-readable storage medium is disclosed. The computer-readable storage medium comprises a plurality of instructions, that when executed by processing circuitry, enable processing circuitry to: display a first image of a first bone segment and a second bone segment; identify a first reduction point on the first bone segment and a second reduction point on the second bone segment, the first and second reduction points to represent a connection point between the first and second bone segments; display a modified first image, the modified first image to collocate the first and second reduction points, the modified first image to display the first bone segment connected to the second bone segment at the collocated first and second reduction points; and receive an indication, the indication representing approval of the modified first image to determine deformity parameters.

In Example 26, the computer-readable storage medium of Example 25, wherein the processing circuitry is further enabled to: display a second image of the first bone segment and the second bone segment, the second image illustrating the first bone segment and the second bone segment at a perspective that is different from a perspective of the first image; identify a third reduction point on the first bone segment; identify a fourth reduction point on the second bone segment, the third and fourth reduction points to represent a second connection point between the third and fourth bone segments; display a modified second image, the modified second image to collocate the third and fourth reduction points, the modified second image to display the first bone segment connected to the second bone segment at the collocated third and fourth reduction points; and receive an indication, the indication representing approval of the modified second image to determine the deformity parameters.

In Example 27, the computer-readable storage medium of Example 26, wherein the processing circuitry is further enabled to resolve a conflict between a deformity parameter common to the modified first image and the modified second image.

In Example 28, the computer-readable storage medium of Example 27, wherein the deformity parameter common to the modified first image and the modified second image comprises an axial translation.

In Example 29, the computer-readable storage medium of Example 25, wherein the processing circuitry is further enabled to: identify a third point on the first bone segment; identify a fourth point on the second bone segment, the third and fourth points to represent a second interconnection point between the first and second bone segments on the first image; and determine a cut line between the first bone segment and the second bone segment based on a line between the second reduction point and the fourth point.

In Example 30, the computer-readable storage medium of Example 29, the modified first image to display a first line between the first reduction point and the third point on the first bone segment.

In Example 31, the computer-readable storage medium of Example 30, the modified first image to display a second line between the second reduction point and the fourth point on the second bone segment.

In Example 32, the computer-readable storage medium of Example 25, wherein identification of a first reduction point on the first bone segment and a second reduction point on the second bone segment comprises identification of two points on the first bone segment, calculation of the midpoint between the two points on the first bone segment, identification of two points on the second bone segment, and calculation of the midpoint between the two points on the second bone segment, wherein the midpoint between the two points on the first bone segment is the first reduction point and the midpoint between the two points on the second bone segment is the second reduction point.

In Example 33, the computer-readable storage medium of Example 25, wherein the first bone segment and the second bone segment comprise two segments of a fractured bone.

In Example 34, the computer-readable storage medium of Example 33, wherein the processing circuitry is further enabled to identify a cut line to separate the first image into a first portion comprising the first bone segment and a second portion comprising the second bone segment.

In Example 35, the computer-readable storage medium of Example 34, wherein the processing circuitry is further enabled to adjust a position of the second portion of the first image to collocate the first reduction point and the second reduction point, to generate the first modified image.

In Example 36, the computer-readable storage medium of Example 25, wherein the processing circuitry is further enabled to adjust a position of a copy of the first image to adjust alignment of the bone segments to create the modified first image.

In one embodiment, Example 37, an apparatus to determine deformity parameters is disclosed. The apparatus comprises: memory and logic circuitry coupled with the memory to enable to the logic circuitry to: display a first image of a first bone segment and a second bone segment; identify a first reduction point on the first bone segment and a second reduction point on the second bone segment, the first and second reduction points to represent a connection point between the first and second bone segments; display a modified first image, the modified first image to collocate the first and second reduction points, the modified first image to display the first bone segment connected to the second bone segment at the collocated first and second reduction points; and receive an indication, the indication representing approval of the modified first image to determine deformity parameters.

In Example 38, the apparatus of Example 37, wherein the logic circuitry is further enabled to: display a second image of the first bone segment and the second bone segment, the second image illustrating the first bone segment and the second bone segment at a perspective that is different from a perspective of the first image; identify a third reduction point on the first bone segment; identify a fourth reduction point on the second bone segment, the third and fourth reduction points to represent a second connection point between the third and fourth bone segments; display a modified second image, the modified second image to collocate the third and fourth reduction points, the modified second image to display the first bone segment connected to the second bone segment at the collocated third and fourth reduction points; and receive an indication, the indication representing approval of the modified second image to determine the deformity parameters.

In Example 39, the apparatus of Example 38, wherein the logic circuitry is further enabled to resolve a conflict between a deformity parameter common to the modified first image and the modified second image.

In Example 40, the apparatus of Example 39, wherein the deformity parameter common to the modified first image and the modified second image comprises an axial translation.

In Example 41, the apparatus of Example 37, wherein the logic circuitry is further enabled to: identify a third point on the first bone segment; identify a fourth point on the second bone segment, the third and fourth points to represent a second interconnection point between the first and second bone segments on the first image; and determine a cut line between the first bone segment and the second bone segment based on a line between the second reduction point and the fourth point.

In Example 42, the apparatus of Example 41, the modified first image to display a first line between the first reduction point and the third point on the first bone segment.

In Example 43, the apparatus of Example 42, the modified first image to display a second line between the second reduction point and the fourth point on the second bone segment.

In Example 44, the apparatus of Example 37, wherein identification of a first reduction point on the first bone segment and a second reduction point on the second bone segment comprises identification of two points on the first bone segment, calculation of the midpoint between the two points on the first bone segment, identification of two points on the second bone segment, and calculation of the midpoint between the two points on the second bone segment, wherein the midpoint between the two points on the first bone segment is the first reduction point and the midpoint between the two points on the second bone segment is the second reduction point.

In Example 45, the apparatus of Example 37, wherein the first bone segment and the second bone segment comprise two segments of a fractured bone.

In Example 46, the apparatus of Example 45, wherein the logic circuitry is further enabled to identify a cut line to separate the first image into a first portion comprising the first bone segment and a second portion comprising the second bone segment.

In Example 47, the apparatus of Example 46, wherein the logic circuitry is further enabled to adjust a position of the second portion of the first image to collocate the first reduction point and the second reduction point, to generate the first modified image.

In Example 48, the apparatus of Example 37, wherein the logic circuitry is further enabled to adjust a position of a copy of the first image to adjust alignment of the bone segments to create the modified first image.

In Example 49, the apparatus of Example 37, wherein the logic circuitry is further enabled to modify image segments of the first image relative to the reduction points, wherein the image segments include a first image segment with the first bone segment and the first reduction point and a second image segment with the second bone segment and the second reduction point, wherein modifications to the image segments translate the first and second reduction points relative to one another.

In Example 50, the computer-readable storage medium of Example 25, wherein the processing circuitry is further enabled to modify image segments of the first image relative to the reduction points, wherein the image segments include a first image segment with the first bone segment and the first reduction point and a second image segment with the second bone segment and the second reduction point, wherein modifications to the image segments translate the first and second reduction points relative to one another.

In Example 51, the method of Example 1, further comprising modifying image segments of the first image relative to the reduction points, wherein the image segments include a first image segment with the first bone segment and the first reduction point and a second image segment with the second bone segment and the second reduction point, wherein modifications to the image segments translate the first and second reduction points relative to one another.

In Example 52, the method of Example 5, wherein the first bone segment and the second bone segment comprise two segments of a fractured or osteotomized bone, wherein a coordinate system of the first image is established by a bone axis overlaid on the first image and image orientation requirements for the first image.

In Example 53, the method of Example 1, wherein the first bone segment and the second bone segment comprise two segments of a fractured or osteotomized bone, wherein a coordinate system of the first image is established by a bone axis overlaid on the first image and image orientation requirements for the first image.

In Example 54, the computer-readable storage medium of Example 25, wherein the first bone segment and the second bone segment comprise two segments of a fractured or osteotomized bone, wherein a coordinate system of the first image is established by a bone axis overlaid on the first image and image orientation requirements for the first image.

In Example 55, the apparatus of Example 37, wherein the logic circuitry is further enabled to create divided images from the first image, the divided images to include a first divided image with the first bone segment and a second divided image with the second bone segment, wherein the divided images can be aligned to collocate the first and second reduction points and one or both of the first divided image and the second divided image are angled to make dividing lines are colinear, wherein the dividing lines are defined between the reduction points and additional points or the dividing lines are directly drawn on the image, one dividing line per bone segment, via input from a user.

In Example 56, the method of Example 1, further comprising creating divided images from the first image, the divided images to include a first divided image with the first bone segment and a second divided image with the second bone segment, wherein the divided images can be aligned to collocate the first and second reduction points and one or both of the first divided image and the second divided image are angled to make dividing lines are colinear, wherein the dividing lines are defined between the reduction points and additional points or the dividing lines are directly drawn on the image, one dividing line per bone segment, via input from a user.

In Example 57, the computer-readable storage medium of Example 25, wherein the processing circuitry is further enabled to create divided images from the first image, the divided images to include a first divided image with the first bone segment and a second divided image with the second bone segment, wherein the divided images can be aligned to collocate the first and second reduction points and one or both of the first divided image and the second divided image are angled to make dividing lines are colinear, wherein the dividing lines are defined between the reduction points and additional points or the dividing lines are directly drawn on the image, one dividing line per bone segment, via input from a user.

In addition, in the foregoing Detailed Description, it can be seen that various features are grouped together in a single example for the purpose of streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels, and are not intended to impose numerical requirements on their objects.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

A data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code to reduce the number of times code must be retrieved from bulk storage during execution. The term “code” covers a broad range of software components and constructs, including applications, drivers, processes, routines, methods, modules, firmware, microcode, and subprograms. Thus, the term “code” may be used to refer to any collection of instructions which, when executed by a processing system, perform a desired operation or operations.

Logic circuitry, devices, and interfaces herein described may perform functions implemented in hardware and also implemented with code executed on one or more processors. Logic circuitry refers to the hardware or the hardware and code that implements one or more logical functions. Circuitry is hardware and may refer to one or more circuits. Each circuit may perform a particular function. A circuit of the circuitry may comprise discrete electrical components interconnected with one or more conductors, an integrated circuit, a chip package, a chip set, memory, or the like. Integrated circuits include circuits created on a substrate such as a silicon wafer and may comprise components. And integrated circuits, processor packages, chip packages, and chipsets may comprise one or more processors.

Processors may receive signals such as instructions and/or data at the input(s) and process the signals to generate the at least one output. While executing code, the code changes the physical states and characteristics of transistors that make up a processor pipeline. The physical states of the transistors translate into logical bits of ones and zeros stored in registers within the processor. The processor can transfer the physical states of the transistors into registers and transfer the physical states of the transistors to another storage medium.

A processor may comprise circuits to perform one or more sub-functions implemented to perform the overall function of the processor. One example of a processor is a state machine or an application-specific integrated circuit (ASIC) that includes at least one input and at least one output. A state machine may manipulate the at least one input to generate the at least one output by performing a predetermined series of serial and/or parallel manipulations or transformations on the at least one input.

While the present disclosure refers to certain embodiments, numerous modifications, alterations, and changes to the described embodiments are possible without departing from the sphere and scope of the present disclosure, as defined in the appended claim(s). Accordingly, it is intended that the present disclosure not be limited to the described embodiments, but that it has the full scope defined by the language of the following claims, and equivalents thereof. The discussion of any embodiment is meant only to be explanatory and is not intended to suggest that the scope of the disclosure, including the claims, is limited to these embodiments. In other words, while illustrative embodiments of the disclosure have been described in detail herein, it is to be understood that the inventive concepts may be otherwise variously embodied and employed, and that the appended claims are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more embodiments or configurations for the purpose of streamlining the disclosure. However, it should be understood that various features of the certain embodiments or configurations of the disclosure may be combined in alternate embodiments, or configurations. Moreover, the following claims are hereby incorporated into this Detailed Description by this reference, with each claim standing on its own as a separate embodiment of the present disclosure.

As used herein, an element or step recited in the singular and proceeded with the word “a” or “an” should be understood as not excluding plural elements or steps, unless such exclusion is explicitly recited. Furthermore, references to “one embodiment” of the present disclosure are not intended to be interpreted as excluding the existence of additional embodiments that also incorporate the recited features.

The phrases “at least one”, “one or more”, and “and/or”, as used herein, are open-ended expressions that are both conjunctive and disjunctive in operation. The terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. All rotational references describe relative movement between the various elements. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

What is claimed is:

1. An apparatus to determine deformity parameters, comprising:
a means for displaying a first image of a first bone segment and a second bone segment;
a means for identifying a first reduction point on the first bone segment and a second reduction point on the second bone segment, the first and second reduction points to represent a connection point between the first and second bone segments;
a means for displaying a modified first image, the modified first image to collocate the first and second reduction points, the modified first image to display the first bone segment connected to the second bone segment at the collocated first and second reduction points; and
a means for receiving an indication, the indication representing approval of the modified first image to determine deformity parameters.

2. The apparatus of claim 1, further comprising:
a means for displaying a second image of the first bone segment and the second bone segment, the second image illustrating the first bone segment and the second bone segment at a perspective that is different from a perspective of the first image;
a means for identifying a third reduction point on the first bone segment;
a means for identifying a fourth reduction point on the second bone segment, the third and fourth reduction points to represent a second connection point between the third and fourth bone segments;
a means for displaying a modified second image, the modified second image to collocate the third and fourth reduction points, the modified second image to display the first bone segment connected to the second bone segment at the collocated third and fourth reduction points; and
a means for receiving an indication, the indication representing approval of the modified second image to determine the deformity parameters.

3. The apparatus of claim 2, further comprising a means for resolving a conflict between a deformity parameter common to the modified first image and the modified second image.

4. The apparatus of claim 3, wherein the deformity parameter common to the modified first image and the modified second image comprises an axial translation.

5. The apparatus of claim 1, further comprising:
a means for identifying a third point on the first bone segment;
a means for identifying a fourth point on the second bone segment, the third and fourth points to represent a second interconnection point between the first and second bone segments on the first image;
a means for determining a cut line between the first bone segment and the second bone segment based on a line between the second reduction point and the fourth point; and
a means to create divided images from the first image, the divided images to include a first divided image with the first bone segment and a second divided image with the second bone segment, wherein the divided images can be aligned to collocate the first and second reduction points and one or both of the first divided image and the second divided image are angled to make dividing lines are colinear, wherein the dividing lines are defined between the reduction points and additional points or the dividing lines are directly drawn on the image, one dividing line per bone segment, via input from a user.

6. The apparatus of claim 1, wherein the means for identifying a first reduction point on the first bone segment and a second reduction point on the second bone segment comprises a means for identifying two points on the first bone segment, a means for calculating a first relative point relative to one or both of the two points on the first bone segment, a means for identifying two points on the second bone segment, and a means for calculating a second relative point relative to one or both of the two points on the second bone segment, wherein the first relative point on the first bone segment is the first reduction point and the second relative point on the second bone segment is the second reduction point.

7. The apparatus of claim 1, wherein the first bone segment and the second bone segment comprise two segments of a fractured or osteotomized bone, wherein a coordinate system of the first image is established by a bone axis overlaid on the first image and image orientation requirements for the first image.

8. The apparatus of claim 7, further comprising a means for identifying a cut line to separate the first image into a first portion comprising the first bone segment and a second portion comprising the second bone segment.

9. A computer-readable storage medium, comprising a plurality of instructions, that when executed by processing circuitry, enable processing circuitry to:
display a first image of a first bone segment and a second bone segment;
identify a first reduction point on the first bone segment and a second reduction point on the second bone segment, the first and second reduction points to represent a connection point between the first and second bone segments;
display a modified first image, the modified first image to collocate the first and second reduction points, the modified first image to display the first bone segment connected to the second bone segment at the collocated first and second reduction points; and
receive an indication, the indication representing approval of the modified first image to determine deformity parameters.

10. The computer-readable storage medium of claim 9, wherein the processing circuitry is further enabled to:
display a second image of the first bone segment and the second bone segment, the second image illustrating the first bone segment and the second bone segment at a perspective that is different from a perspective of the first image;
identify a third reduction point on the first bone segment;
identify a fourth reduction point on the second bone segment, the third and fourth reduction points to represent a second connection point between the third and fourth bone segments;
display a modified second image, the modified second image to collocate the third and fourth reduction points, the modified second image to display the first bone segment connected to the second bone segment at the collocated third and fourth reduction points; and
receive an indication, the indication representing approval of the modified second image to determine the deformity parameters.

11. The computer-readable storage medium of claim 10, wherein the processing circuitry is further enabled to resolve a conflict between a deformity parameter common to the modified first image and the modified second image.

12. The computer-readable storage medium of claim 10, wherein the processing circuitry is further enabled to modify image segments of the first image relative to the reduction points, wherein the image segments include a first image segment with the first bone segment and the first reduction point and a second image segment with the second bone segment and the second reduction point, wherein modifications to the image segments translate the first and second reduction points relative to one another.

13. The computer-readable storage medium of claim 9, wherein the processing circuitry is further enabled to:
identify a third point on the first bone segment;
identify a fourth point on the second bone segment, the third and fourth points to represent a second interconnection point between the first and second bone segments on the first image; and
determine a cut line between the first bone segment and the second bone segment based on a line between the second reduction point and the fourth point.

14. The computer-readable storage medium of claim 9, wherein identification of a first reduction point on the first bone segment and a second reduction point on the second bone segment comprises identification of two points on the first bone segment, calculation of the midpoint between the two points on the first bone segment, identification of two points on the second bone segment, and calculation of the midpoint between the two points on the second bone segment, wherein the midpoint between the two points on the first bone segment is the first reduction point and the midpoint between the two points on the second bone segment is the second reduction point.

15. The computer-readable storage medium of claim 9, wherein the first bone segment and the second bone segment comprise two segments of a fractured or osteotomized bone.
